(19) United States
(12) Patent Application Publication
(10) Pub. No.: US 2024/0350032 A1
(43) Pub. Date: Oct. 24, 2024

(54) Posture-Based Infant Action Recognition System and Method

(71) Applicant: Northeastern University, Boston, MA (US)

(72) Inventors: Sarah OSTADABBAS, Watertown, MA (US); Xiafei Huang, Lynnfield, MA (US); Lingfei Luan, Bowling Green, OH (US); Elaheh Hatamimajoumerd, Boston, MA (US); Micheal Wan, Brunswick, ME (US)

(21) Appl. No.: 18/642,362

(22) Filed: Apr. 22, 2024

Related U.S. Application Data
(60) Provisional application No. 63/461,007, filed on Apr. 21, 2023.

(51) Int. Cl. A61B 5/11; A61B 5/00

(52) U.S. Cl. CPC A61B 5/11; A61B 5/0077; A61B 5/7267; A61B 2503/06

(57) ABSTRACT

Provided herein are methods and systems for recognizing an infant action in a recorded video. The infant action recognition technique includes performing pose estimation for each frame of the video, where pose corresponds to skeletal joint locations and joint angles. A posture classifier uses the pose estimations to classify each pose estimation as one of five postures and a probability value for the posture. The infant action recognition technique further includes using the identified postures for each frame and the probability values to determine a period of uncertainty that corresponds to a transition segment. The infant action recognition technique further includes using the first and last frames of the transition segment to distinguish start and end stable postures. The technique further includes performing filtering and majority voting to remove outlier posture classifications and determine an infant action label for the video based on the start and end stable postures.

### CROSS REFERENCE TO RELATED APPLICATIONS

This application claims the priority of U.S. Provisional Application No. 63/461,007 filed Apr. 21, 2023 and entitled “Posture-Based Infant Action Recognition System and Method”, the whole of which is hereby incorporated by reference.

### STATEMENT REGARDING FEDERALLY SPONSORED RESEARCH OR DEVELOPMENT

This invention was made with government support under Grant No. 2143882 awarded by the National Science Foundation. The government has certain rights in the invention.

### BACKGROUND

Human action recognition from video has become an active area of research in recent years due to advancements in computer vision [20, 47]. Videos may involve human subjects carrying out day-to-day activities in indoor and outdoor settings. While most research has focused on adult subjects—due in part to application objectives such as video surveillance, human-computer interaction, or robotic design—some recent work has centered around children and adolescent subjects, as part of efforts to characterize behavioral or movement disorders [2, 7, 8, 19, 27, 37, 38]. Most recently, infant-domain specific computer vision techniques have been developed to enable further understanding and characterization of infant development [16, 39, 40, 49]. There is a need to extend the benefits of unobtrusive vision-based tools to the domain of video-based infant actions.

Research for pediatric development has consistently shown links between early motor development in infancy and subsequent cognitive, social, and linguistic development in childhood [18, 22]. For instance, as early as six to nine months of age, infant gross motor movements are synchronized with their early vocalizations [17]. Specifically, an infant's babbling is in-rhyme with their limb activity, which may suggest that these movements set the stage for speech development. Links have also been found between poor childhood motor skills and developmental delays, including but not restricted to autism spectrum disorders (ASD) and developmental language conditions [10, 32].

However, the majority of the research has been conducted with school-aged children or adults due to the nature of the tasks that require an understanding of the task instructions. There are some studies that look at infant motor development; nonetheless, this work remains scarce. The implication of video-based action recognition for understanding and characterizing infant development holds immense promise for improving medical diagnoses and treatment plans. Enabling such technology may help identify at-risk infants, assess the effectiveness of behavioral programs, and promote more meaningful caregiver-infant interactions.

In general, video-based human action can be recognized from multiple vision centered modalities, such as appearance [11, 28], depth [6, 23, 33, 43], optical flow [5, 13, 45], and body skeletons [30,35, 41]. Within each modality current state-of-the-art recognition networks have deep structure and require large-scale labeled action datasets with sufficient variations to produce robust performance. Building such datasets of videos is much harder than for images alone, so popular benchmarks for action recognition are smaller in size, having video samples only in the order of 10³ as supposed to 10⁶ in image-based benchmarks. These challenges are magnified in the case of infant action recognition, with no known public dataset of infant actions to date.

Despite the significant progress made in human pose estimation and action recognition, most work is exclusively centered around adult subjects, as evinced by the latest survey published by Institute of Electrical and Electronics Engineers (IEEE) transactions on pattern analysis and machine intelligence (TPAMI) in 2022. Infant action recognition is particularly challenging due to the data scarcity, caused by privacy concerns, as well as high variability in infant movements and difficulty in labeling them by non-experts.

Over the last few years, several approaches to recognizing infant poses have ranged from classical image processing techniques to deep learning-based methods. A domain-adapted infant pose network [14] has been developed from a pre-trained adult pose estimation network, trained and tested on a new image-based dataset, called synthetic and real infant pose (SyRIP). A joint feature coding model [44] was proposed with a ResNet-50 backbone and key point positional encoding to get high-resolution heatmaps of infant poses. However, this model only focuses on infant poses in supine positions. Other research has proposed a deep aggregation vision transformer framework for infant pose estimation [4]. By leveraging a new large-scale infant dataset, called AggPose, with pose labels and clinical labels, a transformer model could detect infant supine position pose from movement frames in video. Additional research has proposed a hierarchical posture classifier based on 3D human pose estimation and scene context information [48]. The research combined ResNet-50, stacked hourglass network, and 3D pose estimation scheme for posture classification, and used estimated 3D keypoints to predict infant postures. Nonetheless, the aforementioned studies have been merely developed for image-based infant pose or posture prediction, and there have been limited studies on infant action recognition. Further research proposed a system called BabyNet to capture infant reaching action [9]. BabyNet uses long short-term memory (LSTM) structure to model motion correlation of different phases of a reaching action, but does not cover other infant action types.

Recently, several infant-specific image/video datasets have been realized, each with its own unique characteristics and applications (see Table 1 for an overview). The babyPose dataset contains over 1000 videos of preterm infants aged between two and six months, captured using a depth-sensing camera along with annotations of 12 limb-joint positions for each frame [26]. However, it only contains the data of newborns with limited supine pose and one-fold background. The SyRIP dataset [14] is an infant pose image dataset including 700 real infant images from YouTube/Google Images and 1000 synthetic infant images generated by rendering skinned multi-infant linear (SMIL) body model with augmented variations in viewpoints, poses, backgrounds, and appearances. In the SyRIP dataset, seventeen joints are annotated for all infant images, and posture labels are also given in four categories (e.g., supine, prone, sitting, and standing) for each real image. Even though this dataset covers various infant poses in the wild, it can only be used to train image-wise models, not for dynamic movement learning such as action or activity recognition. The MINI-RGBD dataset [12] was proposed as a benchmark for a standardized evaluation of pose estimation algorithms in infants. It contains red, green, and blue (RGB) and depth images of infants up to the age of seven months lying in supine position. These images are created by applying SMIL model to build realistic infant body movement sequences with precise 2D and 3D 24 joint positions. The AggPose dataset [4] was proposed to train a deep aggregation transformer for human/infant pose detection. For the dataset, general movements assessment (GMA) devices were adopted to record infant movement videos in supine position.

TABLE 1
An overview of the existing infant-specific image/video datasets used in computer vision tasks.

| | | | Age | | | | |
|---|---|---|---|---|---|---|---|
| Dataset | Content | Purpose | Range | # of Samples | Frame Size | Annotations | Public |
| SyRIP | Real RGB images of [symbol] collected | [symbol] | [symbol] | [symbol] images | Varies | [symbol] 2D & 3D | ✓ |
| [ [symbol] ] | from [symbol] and synthetic RGB [symbol] | | | | | joints location, | |
| | | | | | | 4 [symbol] classes | |
| MINI-RGBD | Synthetic RGB-D videos captured | [symbol] | [symbol] up to | 12 videos | [symbol] | 24 2D & 3D | ✓ |
| [ [symbol] ] | in [symbol] | Motion Analysis | 7 months | ( [symbol] frames) | | joints location | |
| Baby [symbol] | Depth videos of preterm infants in | [symbol] | [symbol] | 16 videos | [symbol] | 12 joints | ✓ |
| [ [symbol] ] | cribs hospitalized in NICU | | | ( [symbol] frames) | | location | |
| [symbol] | [symbol] videos of [symbol] hospitalized to | [symbol] | [symbol] up | 27 videos | [symbol] | [symbol] joints | x |
| [ [symbol] ] | NICU | | to [symbol] | ( [symbol] frames) | | location | |
| [symbol] | [symbol] videos of [symbol] position | [symbol] | [symbol] | [symbol] videos | Unknown | 21 joints | ✓ |
| [ [symbol] ] | | | | ( [symbol] frames) | | location | |
| [symbol] | RGB videos and images of [symbol] | [symbol] Recognition | [symbol] | 200 videos | Varies | [symbol] | ✓ |
| ( [symbol] ) | [symbol] from web | | | ( [symbol] )& 400 images | | | |

[symbol]

indicates data missing or illegible when filed

More than 216 hours of videos and fifteen million frames were extracted. There were 20,748 frames randomly sampled from the videos and professional clinicians annotated the infant 21 keypoints locations. Both MINI-RGBD and AggPose have considerable amounts of data. However, these datasets only include infants performing very simple poses in supine positions, and they can only be employed in newborn pose estimation or behavior analysis. The models trained on these datasets may not have the ability to handle more complicated poses or movements performed by infants, who are learning to roll over, sit down, or stand up. Therefore, the need for a more general infant action dataset is unmet.

### SUMMARY OF THE INVENTION

Described herein are methods and systems for recognizing an infant action in a recorded video. The infant action recognition technique includes performing pose estimation for each frame of the video, where pose corresponds to skeletal joint locations and joint angles. A posture classifier uses the pose estimations to classify each pose estimation as one of five postures. Further, the posture classifier may determine a probability value representing confidence score for each of the five postures. The infant action recognition technique further includes using the identified postures for each frame and the probability values to determine a set of frames that indicate a period of uncertainty. The period of uncertainty thus corresponds to a transition segment where the infant is changing postures. The infant action recognition technique further includes using the first and last frames of the transition segment to distinguish a start stable posture and an end stable posture in the frames of the video. The infant action recognition technique further includes performing filtering and majority voting to remove outlier posture classifications and determine an infant action label for the video based on the start stable posture and the end stable posture.

In one aspect, a computer-implemented method for recognizing an infant action in a video recording is provided. The method includes receiving a video segment of said video recording that captures movement of an infant, wherein the video segment includes a plurality of frames. The method also includes determining, using a posture classification model, posture classification data representing a posture prediction for each frame of the plurality of frames. The method also includes determining a first subset of the plurality of frames representing a transition segment between two stable posture segments, wherein the transition segment includes a first frame in time and a last frame in time. The method also includes determining, based on the posture classification data and the first frame in time of the transition segment, a second subset of the plurality of frames representing a start posture segment. The method also includes determining, based on the posture classification data and the last frame in time of the transition segment, a third subset of the plurality of frames representing an end posture segment. The method also includes determining a start posture label for the start posture segment and determining an end posture label for the end posture segment. The method also includes determining, based on the start posture label and the end posture label, an infant action label for the video segment.

In some embodiments, the method also includes determining probability values corresponding to each frame of the plurality of frames and representing a confidence score for the posture prediction of the corresponding frame. In some embodiments, determining the first subset of the plurality of frames representing the transition segment also includes determining a fourth subset of the plurality of frames representing a period of uncertainty, wherein the probability values of frames corresponding to the fourth subset fail to exceed a threshold value and determining the fourth subset corresponds with the first subset.

In some embodiments, determining the start posture label also includes determining a first stable posture by performing majority voting of the probability values corresponding to the second subset and determining the end posture label also includes determining a second stable posture by performing majority voting of the probability values corresponding to the third subset.

In some embodiments, prior to determining the posture classification, the method also includes determining, using a pose estimation model, pose estimation data representing a human skeleton pose for each frame of the plurality of frames, wherein the human skeleton pose is based on joint locations and joint angles of the infant and the method also includes providing the pose estimation data as input to the posture classification model. In some embodiments, the pose estimation model is trained using an adult pose dataset and an augmented dataset including real-world infant pose data and synthetic infant pose data. In some embodiments, the posture classification model is trained using a two-dimensional infant pose dataset and a three-dimensional infant pose dataset.

In some embodiments, determining the first subset of the plurality of frames representing the transition segment also includes extracting, based on the pose estimation data, a set of feature vectors corresponding to the plurality of frames and determining, using a transition segmentor model with the set of feature vectors as input, the first subset, wherein the transition segmentor model is trained using vectors representing posture transitions. In some embodiments, the set of feature vectors are extracted from a penultimate layer of the posture classification model. In some embodiments, wherein the posture classification model classifies a posture as one of supine, prone, sitting, standing, or all-fours.

In another aspect, a system for recognizing an infant action in a video recording, includes at least one processor and at least one memory in provided. The memory includes instructions that, when executed by the at least one processor, causes the system to receive a video segment of said video recording that captures movement of an infant, wherein the video segment includes a plurality of frames. The instructions further cause the system to determine, using a posture classification model, posture classification data representing a posture prediction for each frame of the plurality of frames. The instructions further cause the system to determine a first subset of the plurality of frames representing a transition segment between two stable posture segments, wherein the transition segment includes a first frame in time and a last frame in time. The instructions further cause the system to determine, based on the posture classification data and the first frame in time of the transition segment, a second subset of the plurality of frames representing a start posture segment. The instructions further cause the system to determine, based on the posture classification data and the last frame in time of the transition segment, a third subset of the plurality of frames representing an end posture segment. The instructions further cause the system to determine a start posture label for the start posture segment and determine an end posture label for the end posture segment. The instructions further cause the system to determine, based on the start posture label and the end posture label, an infant action label for the video segment.

In some embodiments, the instructions further cause the system to determine probability values corresponding to each frame of the plurality of frames and representing a confidence score for the posture prediction of the corresponding frame. In some embodiments, determining the first subset of the plurality of frames representing the transition segment further includes instructions to determine a fourth subset of the plurality of frames representing a period of uncertainty, wherein the probability values of frames corresponding to the fourth subset fail to exceed a threshold value and determine the fourth subset corresponds with the first subset.

In some embodiments, determining the start posture label further includes instructions to determining a first stable posture by performing majority voting of the probability values corresponding to the second subset. In some embodiments, determining the end posture label further includes instructions to determine a second stable posture by performing majority voting of the probability values corresponding to the third subset.

In some embodiments, prior to determining the posture classification, the instructions further cause the system to determine, using a pose estimation model, pose estimation data representing a human skeleton pose for each frame of the plurality of frames, wherein the human skeleton pose is based on joint locations and joint angles of the infant and provide the pose estimation data as input to the posture classification model. In some embodiments, the pose estimation model is trained using an adult pose dataset and an augmented dataset including real-world infant pose data and synthetic infant pose data. In some embodiments, the posture classification model is trained using a two-dimensional infant pose dataset and a three-dimensional infant pose dataset.

In some embodiments, determining the first subset of the plurality of frames representing the transition segment further comprises instructions that cause the system to extract, based on the pose estimation data, a set of feature vectors corresponding to the plurality of frames and determine, using a transition segmentor model with the set of feature vectors as input, the first subset, wherein the transition segmentor model is trained using vectors representing posture transitions. In some embodiments, the set of feature vectors are extracted from a penultimate layer of the posture classification model. In some embodiments, the posture classification model classifies a posture as one of supine, prone, sitting, standing, or all-fours.

In another aspect, a computer-implemented method of generating a dataset of a plurality of infant actions is provided. The method includes receiving a plurality of video recordings that capture actions of human infants. The method also includes determining an infant action label for each video recording of the plurality of video recordings. Determining the infant action label for a video recording, also includes determining a region of interest for each frame of the video recording, wherein the region of interest corresponds to detection of an infant, determining, using the region of interest for each frame, a skeletal pose, determining, using the skeletal pose, a set of skeleton keypoints corresponding to an adult skeleton, and determining, using an action recognition model with the set of skeleton keypoints as input, the infant action label. The method also includes labeling each video of the plurality of video recordings with the infant action label corresponding to the video recording. The method also includes storing the plurality of videos labeled with the infant action label in a database.

In some embodiments, the action recognition model is one of: a recurrent neural network with the skeleton keypoints separated into body part groups, a graph convolutional network with the skeleton keypoints represented as a graph, wherein joints are nodes of the graph and connections between the joints are edges of the graph, and a three-dimensional convolutional network with the skeleton keypoints from each frame converted into a heatmap.

Additional features and aspects of the technology include the following:
1. A computer-implemented method for recognizing an infant action in a video recording, comprising:
   receiving a video segment of said video recording that captures movement of an infant, wherein the video segment includes a plurality of frames;
   determining, using a posture classification model, posture classification data representing a posture prediction for each frame of the plurality of frames;
   determining a first subset of the plurality of frames representing a transition segment between two stable posture segments, wherein the transition segment includes a first frame in time and a last frame in time;
   determining, based on the posture classification data and the first frame in time of the transition segment, a second subset of the plurality of frames representing a start posture segment;
   determining, based on the posture classification data and the last frame in time of the transition segment, a third subset of the plurality of frames representing an end posture segment;
   determining a start posture label for the start posture segment;
   determining an end posture label for the end posture segment; and
   determining, based on the start posture label and the end posture label, an infant action label for the video segment.
2. The computer-implemented method of feature 1, further comprising:
   determining probability values corresponding to each frame of the plurality of frames and representing a confidence score for the posture prediction of the corresponding frame; wherein determining the first subset of the plurality of frames representing the transition segment further comprises:
      determining a fourth subset of the plurality of frames representing a period of uncertainty, wherein the probability values of frames corresponding to the fourth subset fail to exceed a threshold value; and
      determining the fourth subset corresponds with the first subset.
3. The computer-implemented method of feature 2, wherein determining the start posture label further comprises determining a first stable posture by performing majority voting of the probability values corresponding to the second subset;
   wherein determining the end posture label further comprises determining a second stable posture by performing majority voting of the probability values corresponding to the third subset.
4. The computer-implemented method of feature 1, further comprising:
   prior to determining the posture classification, determining, using a pose estimation model, pose estimation data representing a human skeleton pose for each frame of the plurality of frames, wherein the human skeleton pose is based on joint locations and joint angles of the infant; and
   providing the pose estimation data as input to the posture classification model.
5. The computer-implemented method of feature 4, wherein the pose estimation model is trained using an adult pose dataset and an augmented dataset including real-world infant pose data and synthetic infant pose data.
6. The computer-implemented method of feature 4, wherein the posture classification model is trained using a two-dimensional infant pose dataset and a three-dimensional infant pose dataset.
7. The computer-implemented method of feature 4, wherein determining the first subset of the plurality of frames representing the transition segment further comprises:
   extracting, based on the pose estimation data, a set of feature vectors corresponding to the plurality of frames;
   determining, using a transition segmentor model with the set of feature vectors as input, the first subset, wherein the transition segmentor model is trained using vectors representing posture transitions.
8. The computer-implemented method of feature 7, wherein the set of feature vectors are extracted from a penultimate layer of the posture classification model.
9. The computer-implemented method of feature 1, wherein the posture classification model classifies a posture as one of supine, prone, sitting, standing, or all-fours.
10. A system for recognizing an infant action in a video recording, comprising:
   at least one processor; and
   at least one memory including instructions that, when executed by the at least one processor, cause the system to:
      receive a video segment of said video recording that captures movement of an infant, wherein the video segment includes a plurality of frames;
      determine, using a posture classification model, posture classification data representing a posture prediction for each frame of the plurality of frames;
      determine a first subset of the plurality of frames representing a transition segment between two stable posture segments, wherein the transition segment includes a first frame in time and a last frame in time;
      determine, based on the posture classification data and the first frame in time of the transition segment, a second subset of the plurality of frames representing a start posture segment;
      determine, based on the posture classification data and the last frame in time of the transition segment, a third subset of the plurality of frames representing an end posture segment;
      determine a start posture label for the start posture segment;
      determine an end posture label for the end posture segment; and
      determine, based on the start posture label and the end posture label, an infant action label for the video segment.
11. The system of feature 10, wherein the at least one memory further comprises instructions that, when executed by the at least one processor, further cause the system to:
   determine probability values corresponding to each frame of the plurality of frames and representing a confidence score for the posture prediction of the corresponding frame; wherein determining the first subset of the plurality of frames representing the transition segment further includes instructions to:
      determine a fourth subset of the plurality of frames representing a period of uncertainty, wherein the probability values of frames corresponding to the fourth subset fail to exceed a threshold value; and
      determine the fourth subset corresponds with the first subset.
12. The system of feature 11, wherein determining the start posture label further comprises determining a first stable posture by performing majority voting of the probability values corresponding to the second subset;
   wherein determining the end posture label further includes instructions to determine a second stable posture by performing majority voting of the probability values corresponding to the third subset.
13. The system of feature 10, wherein the at least one memory further comprises instructions that, when executed by the at least one processor, further cause the system to:
   prior to determining the posture classification, determine, using a pose estimation model, pose estimation data representing a human skeleton pose for each frame of the plurality of frames, wherein the human skeleton pose is based on joint locations and joint angles of the infant; and
   provide the pose estimation data as input to the posture classification model.
14. The system of feature 13, wherein the pose estimation model is trained using an adult pose dataset and an augmented dataset including real-world infant pose data and synthetic infant pose data.
15. The system of feature 13, wherein the posture classification model is trained using a two-dimensional infant pose dataset and a three-dimensional infant pose dataset.
16. The system of feature 13, wherein determining the first subset of the plurality of frames representing the transition segment further comprises instructions that, when executed by the at least one processor, further cause the system to:
   extract, based on the pose estimation data, a set of feature vectors corresponding to the plurality of frames;
   determine, using a transition segmentor model with the set of feature vectors as input, the first subset, wherein the transition segmentor model is trained using vectors representing posture transitions.
17. The system of feature 16, wherein the set of feature vectors are extracted from a penultimate layer of the posture classification model.
18. The system of feature 10, wherein the posture classification model classifies a posture as one of supine, prone, sitting, standing, or all-fours.
19. A computer-implemented method of generating a dataset of a plurality of infant actions, comprising:
   receiving a plurality of video recordings that capture actions of human infants;
   determining an infant action label for each video recording of the plurality of video recordings, wherein determining the infant action label for a video recording further comprises:
      determining a region of interest for each frame of the video recording, wherein the region of interest corresponds to detection of an infant;
      determining, using the region of interest for each frame, a skeletal pose;
      determining, using the skeletal pose, a set of skeleton keypoints corresponding to an adult skeleton; and
      determining, using an action recognition model with the set of skeleton keypoints as input, the infant action label;
   labeling each video of the plurality of video recordings with the infant action label corresponding to the video recording; and
   storing the plurality of videos labeled with the infant action label in a database.
20. The computer-implemented method of feature 19, wherein the action recognition model is one of:
   (a) a recurrent neural network with the skeleton keypoints separated into body part groups;
   (b) a graph convolutional network with the skeleton keypoints represented as a graph, wherein joints are nodes of the graph and connections between the joints are edges of the graph; and
   (c) a three-dimensional convolutional network with the skeleton keypoints from each frame converted into a heatmap.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the infant action recognition method, as described herein. The infant action recognition method includes three components: posture prediction, transition segmentation, and action recognition. Contents in the dotted boxes indicate intermediate outputs.

FIG. 2 illustrates video examples of the InfAct dataset. Each video example is segmented by several frames to illustrate a start posture segment, a transition segment, and an end posture segment. FIG. 2 illustrates the five posture classes: supine, prone, sitting, standing, and all-fours.

FIG. 3A illustrates, in a bar graph, descriptive statistics of InfAct dataset action classes distribution. The nine most common action classes are used for infant action recognition.

FIG. 3B illustrates a duration analysis of video sequences for determining the InfAct dataset.

FIG. 4 illustrates the pose-based posture classification performance. For each infant action example, the predicted 2D pose is overlaid on an original image, while the predicted 3D body mesh is overlaid on the cropped image. Each example is labeled with three boxes, where the first box represents the ground truth label, the second box represents the 2D pose-based posture prediction, and the third box represents the 3D pose-based result. Shaded boxes represent incorrect predictions.

FIG. 5 illustrates confusion matrices of infant 2D pose-based and 3D pose-based posture classification models before and after fine-tuning using the InfAct training images.

FIG. 6 illustrates examples of predicted infant posture probability signals and corresponding estimated transition segmentation results. The vertical lines indicate the predicted index of the last frame of the start posture and the predicted index of the first frame of the end posture respectively, while the dashed vertical lines indicate the ground truth.

FIG. 7 illustrates example images of the InfActPrimitive dataset. Each row corresponds to one of the five infant primitive action classes (e.g., supine, prone, sitting, standing, and all-fours) of the dataset.

FIG. 8 illustrates a bar graph of the frequency of each action class collected from both the YouTube platform and recruited participants through an approved experiment.

FIG. 9 illustrates a schematic of the overall infant action recognition pipeline, encompassing infant-specific preprocessing and the action recognition phase. The infant is initially detected in raw frames using You Only Look Once Version 7 (YOLOv7) and subsequently serves as input for both 2D and 3D pose estimation facilitated by fine-tuned domain-adapted infant pose (FiDIP) model and heuristic weakly supervised 3D human pose estimation infant (HW-HuP-Infant) model, respectively. The resulting pose information may be further processed into heatmaps, serving as input for convolutional neural network (CNN)-based models, or represented as graphs or sequences for graph—and recurrently neural network (RNN)-based models to predict infant actions.

FIG. 10 illustrates three distinct skeleton layouts employed in skeleton-based action recognition datasets. The adult skeleton data adheres to the NTU RGB+D layout, while the 3D version of InfActPrimitive adopts the Human3.6M layout. Action recognition models utilize the common keypoints shared between these layouts. Additionally, both the 2D versions of adult and infant skeleton data conform to the common objects in context (COCO) layout.

FIG. 11 illustrates classification results of three models, along with their respective confusion matrices. As shown, an InfoGCN model faces challenges in achieving clear distinctions between classes, whereas the other models demonstrate varying degrees of proficiency in classifying different primitive categories.

FIG. 12 illustrates 2D latent projections generated through t-SNE for validation samples from both the NTU RGB+D and InfActPrimitive datasets. The results, presented from left to right, demonstrate the projection of the latent variables produced by PoseC3D, InfoGCN, and ST-GCN. While these methods effectively capture patterns in adult actions within the NTU RGB+D dataset, they struggle to distinguish between infant actions in the InfActPrimitive dataset.

### DETAILED DESCRIPTION OF THE INVENTION

Automatic detection of infant actions from home videos and videos recording in a clinical or educational setting could aid medical and behavioral specialists in the early detection of motor impairments in infancy. However, most computer vision approaches for action recognition are centered around adult subjects, following datasets and benchmarks in the field. The present technology provides a data-efficient pipeline for infant action recognition based on modeling an action as a time sequence consisting of two different stable postures with a transition period between them. The postures are detected frame-wise from the estimated 2D and 3D infant body poses, while the action sequence is segmented based on the posture-driven low-dimensional features of each frame. An infant action dataset named InfAct is provided and consists of 200 fully annotated home videos representing a wide range of common infant actions. Among the ten more common classes of infant actions, the action recognition model achieved 78.0% accuracy when tested on InfAct.

Using postures as low-dimensional representations of actions allows the model to be conservative with data usage during supervised steps of the model training. A video segmenter is provided to detect the onset and offset of the transition state and then using the segmentation results and the frame-wise posture-based probabilities, the action label can be determined.

Alongside creating InfAct, a data-efficient pipeline for infant action recognition is provided that robustly detects actions given a limited number of samples for each category of common action. The InfAct dataset advances the field of video-based infant action recognition by offering a more accurate and objective mechanism to assess infant motor development. The 200 fully annotated home videos show the potential of video-based infant action recognition for motor development monitoring.

The present technology allows parents to directly observe their child and provides alerts when an action occurs. The visual triggers in previous such systems are often simple “motion within zone” triggers and have many false alarms as well as fail to catch events of interest, which can be identified by the present technology, including (1) lethal or otherwise serious injuries to the infant caused by preventable accidents; (2) missing early signs of neurodevelopmental disorders including torticollis, cerebral palsy (CP), autism spectrum disorder (ASD); and (3) sudden infant death syndrome (SIDS) during sleep.

The present technology utilizes a novel infant action recognition algorithm that deals with data limitations by representing each infant action as a sequence of a start posture state to a transition state to an end posture state. These postures are the main milestone positions that an infant takes in the first year of their life, defined by the Alberta infant motor scale (AIMS) [29]. Using postures as low-dimensional representations of actions allows the model to be conservative with data usage during supervised steps of the model training. A video segmenter is used to detect the onset and offset of the transition state, and then the segmentation results and the frame-wise posture-based probabilities are used to determine the action label. Also provided is a novel, curated infant action dataset, called InfAct, containing 200 infant videos, with accurate posture state and transition segment annotations.

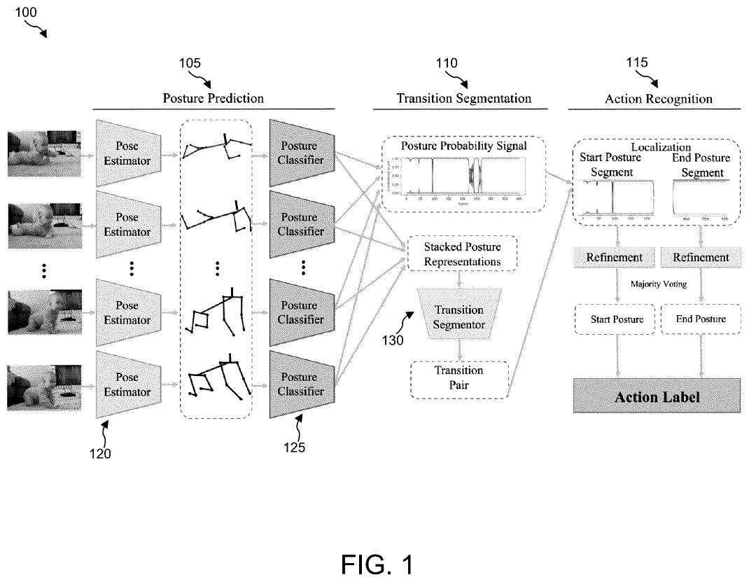

In general, human action recognition aims to understand human behavior by assigning labels to the actions present in a given video. In the infant behavior domain, common actions related to infant motor development milestones include rolling, sitting down, standing up, etc. The methods and techniques described herein introduce a data-efficient infant recognition model alongside an infant in-the-wild action dataset containing annotated video of infant actions, each clipped to feature a single transition between initial and final periods of stable postures (e.g., sitting→sit-to-stand transition→standing). A three-part process, illustrated in FIG. 1, that has the following components: (1) a posture prediction component 105 that includes a pose-based infant posture classification model 125 which produces frame-wise posture predictions (and associated probabilities), (2) transition segmentation component 110 that includes a transition segmentation model (e.g., transition segmentor 130) which is trained to predict the start and end times of periods of posture transition (between periods of stable posture), and (3) action recognition component 115 that includes an action recognition model, which classifies postures in each of the stable posture periods before and after the transition, by smoothing posture prediction probability signals, and then produces a final action label based on those predicted postures.

As shown in FIG. 1, the infant action recognition process includes three components: the posture prediction component 105, the transition segmentation component 110, and action recognition component 115. At the posture prediction component 105, a video of an infant action is received and analyzed frame by frame. For each frame, an infant pose estimator 120 is used to predict the pose of the infant at each frame of the video. A pose represents the skeletal joint locations and joint angles. The resulting pose estimation data (i.e., pose estimation for each frame) is then provided as input to the pose-based infant posture classification model 125. The posture classification model 125 determines a posture for the pose estimation of each frame each frame, as well as a probability value representing a confidence score for the determined posture. In some embodiments, the posture classification model 125 determines a probability value for each posture classification.

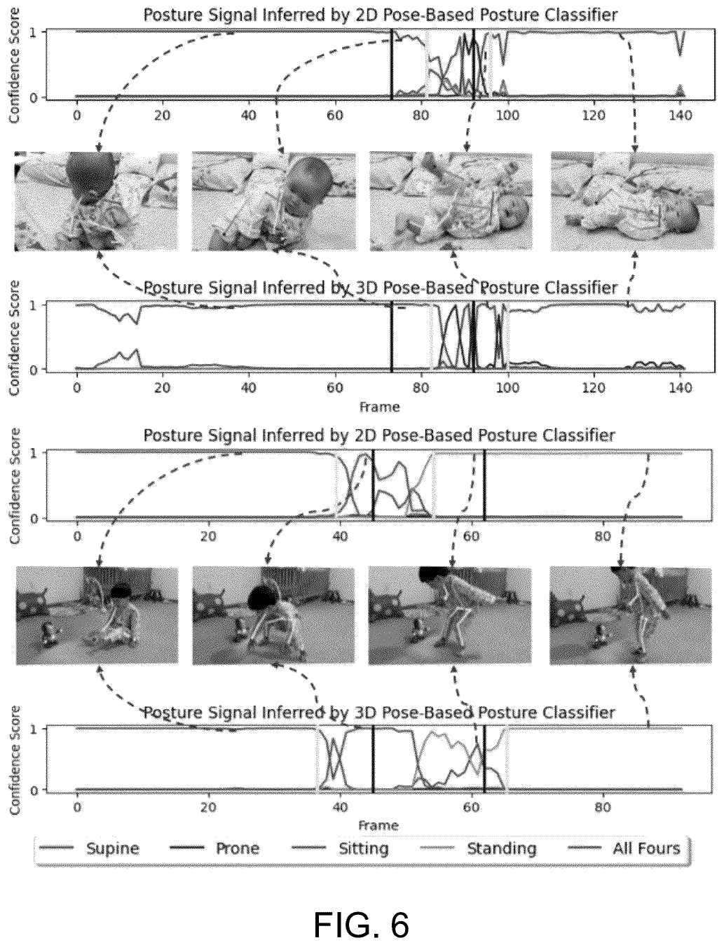

At the transition segmentation component 110, the probability values for the frames of the video are used to generate a posture probability signal. As shown in FIG. 6, the probability values (e.g., confidence scores) for each of the posture classifications create a posture probability signal for the respective posture classification. The posture probability signals may be analyzed to determine a segment (e.g., a set of frames) where none of the posture probability signals (e.g., the probability values) are high. In some embodiments, the transition segmentation component 110 may identify the segment by determining none of the posture probability signals exceed a predetermined threshold value. This determined segment may be identified as a transition segment that is a first and last frame, which are used to distinguish the transition segment from frame segments that precede and follow the transition segment.

Additionally, the transition segment may be determined using a transition segmentor model 130. The transition segmentor model 130 may be trained using feature vectors that represent transitional frames. The transition segmentor model 130 may then be used to identify frames representing transitional frames based on the feature vector corresponding to the frame. For each frame of the video, a feature vector may be obtained from the last layer of the posture classification model 125 when determining the posture classification for the particular frame. The transition segmentor model 130 may receive the feature vectors as input and determine the feature vectors, and thus the corresponding frames, that represent a transition segment.

The transition segmentation component 110 may use the transition segment to determine the stable postures (e.g., a start posture and an end posture) that precede and follow the transition segment, based on the predominant posture classification of the frames that precede and follow the frames of the transition segment. The determined start posture and end posture may be identified as a transition pair.

Finally, the action recognition component 115 may determine the action label based on the start posture and end posture. The action recognition component 115 may perform localization by removing the posture probability data corresponding to the frames of the transition segment from the posture probability signals. Removing the posture probability data corresponding to the frames of the transition segment may result in two sets of frames (e.g., start posture and end posture), with corresponding posture probability data. The action recognition component 115 may perform refinement of the two sets of frames, such as moving average techniques to smooth any short-term anomalies or fluctuations. Further, the action recognition component 115 may perform majority voting for the data of the two sets of frames to finally determine the two posture classes corresponding to the frames occurring before and after the transition segment of frames. The determined two posture classes (e.g., the start posture and the end posture) are then used to as part of the action label that labels the provided video segment.

Pose and posture recognition may not be perfectly accurate, thus infant domain knowledge is applied by adapting the model to the five infant posture classes: Supine, Prone, Sitting, Standing, and All-fours. Another aspect of utilizing domain knowledge is that outlier posture identifications may be removed. For example, if the pose-based infant posture classification model 125 determines there are fifty frames of an infant standing, then one frame of the infant in prone position, then followed by another twenty frames of the infant standing, then it is understood that for one frame the infant in real life did not suddenly go from a standing position into a prone position and then back to standing. Thus, a posture classification that is determined to be an outlier may be removed.

### Problem Formulation

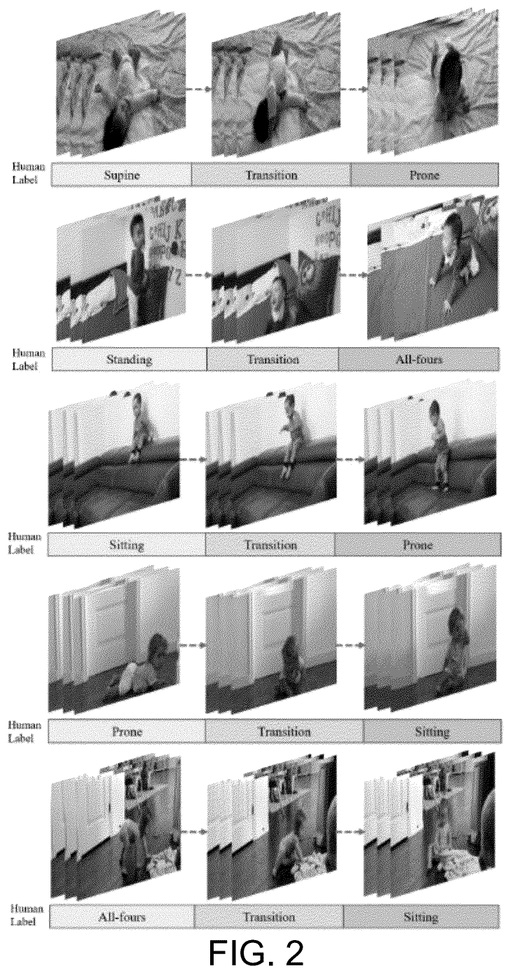

An infant action may be conceptualized as a change from one stable posture to another stable posture with a transition period in between, with stable postures defined as those lasting at least one second. Some samples following on this schema are shown in FIG. 2. Formally, a video X is represented as sequence of T image frames, X=(x¹, . . . , xT). The infant action label of the video takes the form of A=(p^{s}, p^{e}), where p^{s}, p^{e}∈{Supine, Prone, Sitting, Standing, All-fours} are the stable start and end postures, respectively. These five critical atomic posture classes are taken from the Alberta infant motor scale (AIMS) guideline [29]. Assuming that p^{s}≠p^{e}, there are 20 possible action classes based on the posture combinations. For given action A, the transition period between stable postures is given by Y=(y^{s}, y^{e}), with y^{s} the index of the last frame of the start posture p^{s}, and y^{e}>y^{s} the index of the first frame of the end posture p^{e}.

### Posture Prediction

As shown in FIG. 1, the infant action recognition method 100 may include a posture prediction component 105 that employs an infant pose estimator 120 to predict the pose of the infant at each frame of the video, and then according to the inferred poses, utilizes an infant pose-based posture classifier 125 to estimate the series of infant postures. A classification method, such as the appearance independent posture classification method [15], may be used to determine a posture prediction. The appearance independent posture classification method may be modified for each frame x^{t} of the action video sequence X to obtain a posture prediction p^{t}, for t∈{1, . . . , T}. The appearance independent posture classification method first extracts either a 2-dimensional (2D) or 3-dimensional (3D) human skeleton pose prediction J^{t}∈R^{N×D}, where N=12 is the number of skeleton joints (corresponding to the shoulders, elbows, wrists, hips, knees, and ankles), and D∈{2, 3} is spatial dimension of the coordinates. Underlying pose estimators 120, such as the fine-tuned domain-adapted infant pose (FiDIP) model for 2D and the heuristic weakly supervised 3D human pose estimation infant (HW-HuP-Infant) model [25] for 3D, are specifically adapted for the infant domain. Then the pose J^{t} is fed into a 2D or 3D pose-based posture classifier, resulting in the posture prediction p^{t}. The original posture classification model, such as appearance independent posture classification method [15], produces one of four posture classes, so this model's network is retrained using images representing the five classes extracted from the synthetic and real infant pose (SyRIP) dataset [14].

### Transition Segmentation

As shown in FIG. 1, the infant action recognition method 100 may include a transition segmentation component 110 that uses an infant transition segmentor 130 to extract start and end stable posture segments. To predict the frame indices of the transition period, Y=(y^{s}, y^{e}), a speech sequence segmentation model may be adapted. As input, the underlying feature vectors p̅=(p̅¹, . . . , p̅^{T}) are taken from the last layer of the posture estimation model. The datapoint p̅ is used to train the speech sequence segmentation model, a bi-directional recurrent neural network (Bi-RNN), supervised by the ground truth label Y=(y^{s}, y^{e}). During training, the model searches through possible start and end transition timings to minimize the loss function measuring a distance between the predicted transition state \tilde{Y} and ground truth label Y.

### Action Recognition

As shown in FIG. 1, the infant action recognition method 100 may include an action recognition component that identifies action labels for the entire video clip based on the start and end posture labels of the corresponding segments after refinement and majority voting on the posture probability signals. For the start, transition, and end segments identified, the task is to predict the posture classes of the stable start and end segments, which together entail the overall predicted action class. The transition segmentation prediction yields frame indices Y=(y^{s}, y^{e}), and from these the sub-sequences of posture predictions may be derived for the start and end stable posture periods, (p¹, . . . , p^{y}^{^{s}}) and (p^{y}^{ₑ}, . . . , p^{T}), respectively. Different moving average techniques may be applied to smooth out short-term fluctuations and highlight longer-term trends [46], and obtain smoothed posture sequences (\hat{p}¹, . . . , \hat{p}^{y}^{^{s}}) and (\hat{p}^{y}^{ₑ}, . . . , \hat{p}^{T}). Then these sequences are aggregated with majority voting to produce final class estimations A=(\hat{p}^{s}, \hat{p}^{e}).

### An Infant Action Dataset

A specialized infant action dataset may be produced to enable research in computer vision infant action comprehension, and to provide a testbed for infant action recognition algorithms. The specialized infant action dataset, referred to as InfAct, may comprise video clips of infant activities and images of infant postures, with structured action and transition segmentation labels. FIG. 2 illustrates the form of the video data, which contain transitions from a stable starting posture to a stable ending posture.

The video sourcing and selection procedure may be developed by an experienced psychologist. The video sourcing and selection methodology may be include a comprehensive search of public videos, such as from YouTube, to obtain a representative cross-section of infant postures and actions, and to ensure the inclusion of a wide range of both infant-specific and general characteristics, including apparent race and ethnicity, stable and transitional postures, and environmental settings. A stringent selection criteria may be applied to ensure that postures and transitions are represented consistently and with sufficient duration. After selection, videos may be pre-processed and clipped to yield a final set of short videos depicting a transition between a stable starting posture and a stable ending posture, with broad representation of postures on both ends, and movements in the transition stage. Finally, the resulting action clips may be annotated with start and end time stamps for the transition period, and labels for the posture classes in the initial and final stable posture periods.

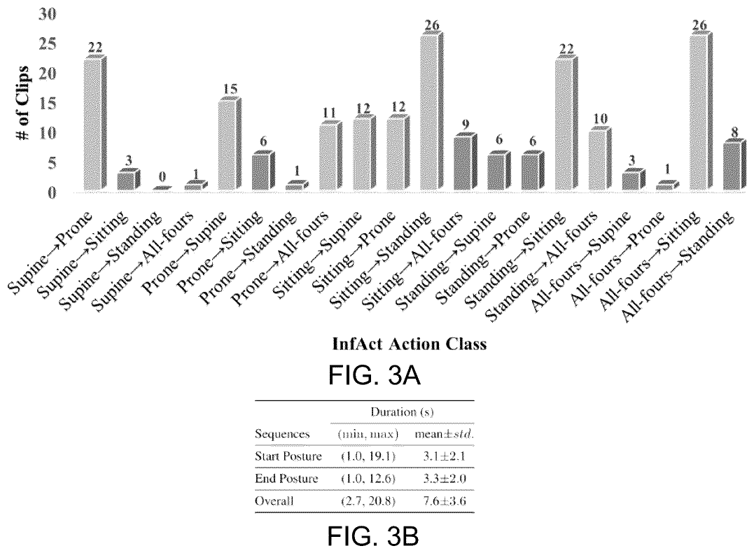

In an example sample set of the source video titles, a visual inspection and evidence provided an estimate that infants in the InfAct dataset range in age from 3 to 18 months. The video clip resolutions vary from 720×576 to 1280×720 pixels. The environments of the recordings vary, to include living rooms, bedrooms, outdoors, bathrooms, the kitchen, and the playground. FIG. 3 shows the statistical analysis of a sample InfAct dataset.

### Experimental Results

The InfAct dataset may be used to evaluate the performance of three model components, including posture classification 105, transition segmentation 110, and action recognition 115 as shown in FIG. 1.

### Datasets

To evaluate the action recognition model, video samples having improbable actions were excluded from the InfAct dataset, resulting in videos across the nine action classes highlighted in FIG. 3. The InfAct videos may be used to train the transition segmentation model (e.g., transition segmentor 130). A posture dataset of images may be created by extracting one frame at the beginning and end of each video in InfAct, to define a 300-100 train-test split. Furthermore, real infant images from the SyRIP dataset may be re-defined with the present five posture classes (modified from the existing four), to define a 600-100 train-test split.

### Pose-based Posture Classification

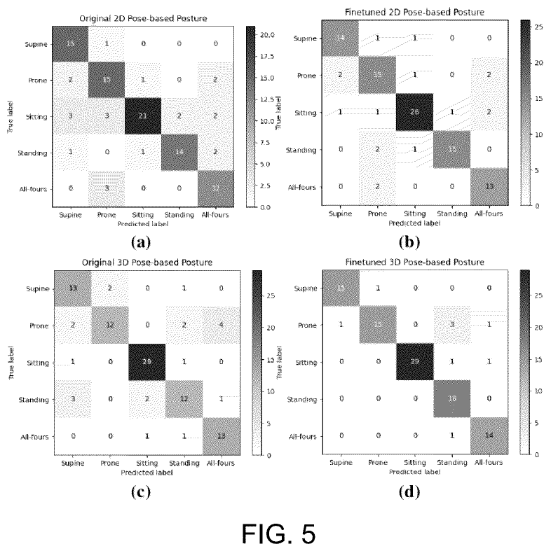

First, both the 2D and 3D pose-based posture classification networks may be defined using the SyRIP dataset (e.g., 400 epochs, Adam optimizer, learning rate of 0.00006) that uses a network with four fully connected layers [15]. The trained network may then be fine-tuned with additional InfAct training image (e.g., 10 epochs, learning rate of 0.001, batch size of 50). The posture prediction accuracy scores may be reported for both the initial model trained on SyRIP and the fine-tuned model trained further on InfAct, as shown in Table 2. These example results show that fine-tuning on InfAct notably improves performance, as does adopting the 3D posture model. The fine-tuned 3D pose-based posture model reaches a high overall accuracy of 91.0%. The corresponding prediction confusion matrices shown in FIG. 5 also attest to strong performance. The results also reveal a higher-than-typical confusion between the prone and all-fours postures, possibly due to the similarity of these poses, or simply the limited availability of training data.

TABLE 2
Performance of the five-posture classification models trained on SyRIP
and fine-tuned posture models on InfAct test set in accuracy.

| | | Posture Accuracy (%) | | | | | |
|---|---|---|---|---|---|---|---|
| Model | Posture Model | Average | Supine | Prone | Sitting | Standing | All-fours |
| 2D | Trained on SyRIP | 77.0 | 93.8 | 75.0 | 67.7 | 77.8 | 80.0 |
| | Fine-tuned on InfAct | 83.0 | 87.5 | 75.0 | 83.9 | 83.3 | 86.7 |
| 3D | Trained on SyRIP | 79.0 | 81.3 | 60.0 | 93.6 | 66.7 | 86.7 |
| | Fine-tuned on InfAct | 91.0 | 93.8 | 75.0 | 93.6 | 100.0 | 93.3 |

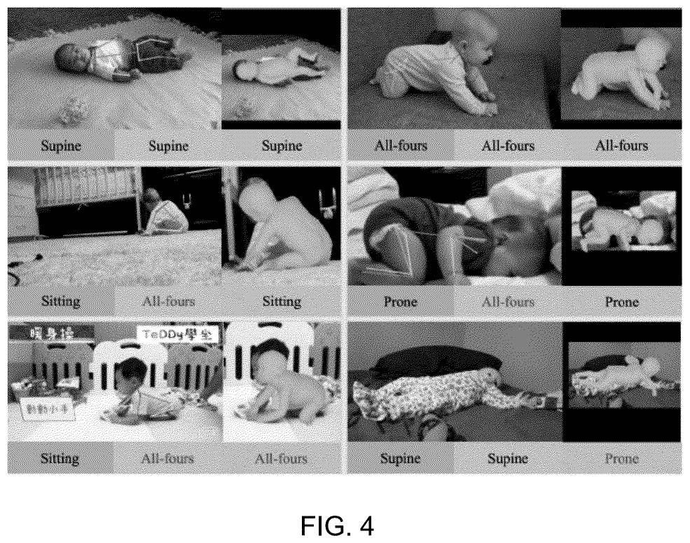

Visualizations of pose and posture predictions are shown in FIG. 4. The first row shows examples in which the posture is correctly predicted with both 2D and 3D pose information as inputs. In examples in the second row, the 3D pose-based posture prediction model succeeds while the 2D pose-based model fails, and in the third row, 3D models fail because predicted 3D poses are wrong. The better performance of the 3D pose-based model could be due to the underlying 3D pose estimations being more robust across a variety of camera angles, resulting in more reliable posture estimations.

### Posture-based Transition Segmentation

The transition segmenter 130 consists of two bidirectional LSTM layers, each followed by a dropout layer, and is well-suited to handle variable-length sequences. This network may be trained on InfAct data with the following configurations of input derived from the preceding posture estimation model (e.g., posture classifier 125).

Posture Probabilities: For each frame, a vector of five probabilities from the posture estimation model (e.g., posture classifier 125) corresponding to each of the five posture classes. The input dimension was L×C for a sequence of length L and C=5 classes (e.g., Supine, Prone, Sitting, Standing, All-fours).

Joint Locations: For each frame, a residual vector obtained by applying principle components analysis (PCA) [31] to the sequence of keypoint coordinates for each body joint. The PCA reduction converts coordinate vectors of 17×2 or 17×3 dimensions, depending on the spatial dimension, down to K=10 dimensions, for an overall input dimension of L×K for a sequence of length L.

Posture Features: For each frame, a residual vector obtained by applying PCA to the feature vector representation of the image in the penultimate layer of the posture estimation model (e.g., posture classifier 125). The PCA reduction converts coordinate vectors down from 16 to K=10 dimensions, for an overall input dimension of L×K for a sequence of length L.

The model may be trained with the Adam optimizer at a learning rate of 0.01, with batch size 10. Following the original speech segmentation model [1], the loss for a prediction Y=(\tilde{y}^{s}, \tilde{y}^{e}) relative to the ground truth Y=(y^{s}, y^{e}) is given by the structured loss: [symbol] (Y, \tilde{Y})=Σ_{i=s,e} max (0, ∥yⁱ−y⁻ⁱ∥τ) with units in frames, and τ=5 frames is a tolerance factor to allow for natural variations in human annotation. The video framerate was 25 Hz, and each frame was used in the input to the transition segmentation model (e.g., transition segmentor 130). Test results for the transition segmentation model (e.g., transition segmentor 130) based on structured loss are shown on the left side of Table 3. The results show that, under both the 2D and 3D paradigms, transition segmentation estimation performance is stronger when posture estimation model (e.g., posture classifier 125) features (such as classification probabilities or last layer features) are used as input, compared to the raw joint locations. In the present conceptual framework, and in the InfAct dataset, the notion of the transition period is heavily tied to the notion of posture, which the posture estimation model (e.g., posture classifier 125) is trained to reason about. This is clear in the visualizations presented in FIG. 6, where posture probabilities, transition segments, and video frames are aligned; transitions are strongly correlated with periods of posture prediction uncertainty.

TABLE 3
Performance of transition segmentation
models on InfAct test videos.

Structured Loss

| Posture Estimation | Input Sequence | Frames | s |
|---|---|---|---|
| 2D Pose-based | Posture Probabilities | 39.0 | 1.6 |
| | Joint Locations | 58.5 | 2.3 |
| | Posture Features | 37.7 | 1.5 |
| 3D Pose-based | Posture Probabilities | 39.2 | 1.6 |
| | Joint Locations | 52.8 | 2.1 |
| | Posture Features | 36.5 | 1.5 |

The results also show that using 3D pose-based posture model features (either model probabilities or last layer features) as input boosts performance over 2D pose-based posture features, but interestingly this advantage is erased when joint locations alone are used as input. The strongest model, which uses 3D pose-based posture model features as input, has an average structured loss of 36.5 frames or ˜1.5 s, which is reasonable relative to human perception.

### Posture-based Action Recognition

The final step in the process is to predict posture classes in the starting and ending stable posture periods, and thus infer the final action class label. The posture prediction is based on majority voting of the predicted posture class over the two stable posture periods, with start and end timestamps for those stable periods determined by the preceding temporal segmentation model. For the test results, the posture estimation model (2D or 3D) and the transition segmentation input format (posture model probabilities, joint coordinate locations, or last-layer posture model features) were varied, and also the transition segment determined by the ground truth was tested, for reference. Furthermore, while the majority voting is always based on the sequence of predicted posture classes (regardless of which sequence of posture features is fed into the transition segmentation model (e.g., transition segmentor 130)), two methods of smoothing this sequence to stabilize the raw signal were tested. In particular, a moving average (MA) and an exponentially weighted moving average (EWMA) with a fixed window size of five frames were tested. Taken together, the smoothing and subsequent majority voting produce a single class label for each of the starting and ending stable postures, from which a single overall action class can be inferred for each video clip. The classification accuracy of this final action class label against the ground truth label, for each of the methodological variations considered, is tabulated in Table 4. A correct prediction requires that both the starting and ending stable class posture be correctly identified, highlighting the roughly “squared” difficulty of the prediction task.

TABLE 4
Performance of the infant action recognition method 100 on InfAct test set with
different kinds of input sequences by applying different refinement methods.

| | | | | | Raw | MA | EWMA |
|---|---|---|---|---|---|---|---|
| Posture Estimation | Posture Feature | Transition Segment | | | Acc. (%) | Acc. (%) | Acc. (%) |
| 2D Pose-based | Posture Preds. | Pred. from | | Posture Probs. | 64.0 | 66.0 | 66.0 |
| | | | {open oversize brace} | Joint Locs. | 54.0 | 54.0 | 54.0 |
| | | | | Posture Feats. | 62.0 | 66.0 | 66.0 |
| | | Ground Truth | | | 70.0 | 72.0 | 74.0 |
| 3D Pose-based | Posture Preds. | Pred. from | | Posture Probs. | 72.0 | 72.0 | 72.0 |
| | | | {open oversize brace} | Joint Locs. | 60.0 | 62.0 | 62.0 |
| | | | | Posture Feats. | 78.0 | 78.0 | 80.0 |
| | | Ground Truth | | | 86.0 | 86.0 | 86.0 |

On the whole, the results track and were largely determined by the performance of the underlying transition segmentation model (e.g., transition segmentor 130), with segmentation based on 3D pose-based posture estimation coming out on top. 3D-based transition segmentation may yield better results than 2D, as does posture model-based sequential input for transition segmentation compared to joint coordinate location sequential input. The extent to which improvements in segmentation results lead to improvements in action recognition is remarkable-a structured loss delta of ˜0.8 s between the best and worst segmentation performances yields up to a 24 percentage point gain in action recognition, to 78.0%. Using the ground truth segmentation labels bumps performance further to 86.0%. This may be explained in part by the statistical effect alluded to earlier, wherein the action recognition accuracy is roughly the square of the stable posture estimation accuracy, so improvements in segmentation leading to improvements in stable posture estimation are magnified for final action recognition. The performances of the different smoothing methods were much more balanced, with results slightly favoring the EWMA.

The determination of the InfAct dataset described above includes its own challenges. Further described below are methods and techniques for the determination of an InfAct dataset, such as determining an InfActPrimitive dataset. The InfActPrimitive dataset encompasses five significant infant milestone action categories and the determination of the InfActPrimitive dataset may incorporate specialized preprocessing for infant data.

Automated human action recognition is a rapidly evolving field within computer vision, finding wide-ranging applications in areas such as surveillance, security [72], human-computer interaction [60], tele-health [70], and sports analysis [77]. In healthcare, especially concerning infants and young children, the capability to automatically detect and interpret their actions holds paramount importance. Precise action recognition in infants serves multiple vital purposes, including ensuring their safety, tracking developmental milestones, facilitating early intervention for developmental delays, enhancing parent-infant bonding, advancing computer-aided diagnostic technologies, and contributing to the scientific understanding of child development.

The notion of action in the research literature exhibits significant variability and remains a subject of ongoing investigation [68]. Determination of the dataset is primarily focused on recognizing infants' fundamental motor primitive actions that may encompass five posture-based actions (e.g., sitting, standing, supine, prone, and all-fours) as defined by the AIMS [54]. These actions correspond to significant developmental milestones achieved by infants in their first year of life.

In some embodiments, to facilitate accurate recognition of these actions, skeleton-based models may be employed, which are notable for their resilience against external factors like background or lighting variations. In comparison to RGB-based models, these skeleton-based models offer superior efficiency. Given the skeleton-based models ability to compactly represent video data using skeletal information, these models prove to be especially useful in situations where labeled data is scarce. Therefore, the employment of the skeleton based models enables a more efficient recognition of the aforementioned hierarchy of infant actions, even with a small data pool [64].

While state-of-the-art skeleton-based human action recognition and graphical convolution network (GCN) models [61, 79] have achieved impressive performance, they are primarily focused on the adult domain and relied heavily on large, high-quality labeled datasets. However, there exists a significant domain gap between adult and infant action data due to differences in body shape, poses, range of actions, and motor primitives. Additionally, even for the same type of action, there are discernible differences in how the action is performed by the body between infants and adults. For example, a sitting action for adults often involves the use of a chair or an elevated surface, which provide stability and support, while infants when performing a sitting action typically sit on the floor, relying on their developing core strength and balance, which results in different skeleton representations than adults. Furthermore, adult action datasets like “NTU RGB+D” [71] and “N-UCLA” [75] primarily include actions such as walking, drinking, and waving, which do not involve significant changes in posture. In contrast, infant actions like rolling, crawling, and transitioning between sitting and standing may include distinct postural transitions. This domain gap poses significant challenges and hampers the current models' ability to accurately capture the complex dynamics of infant actions.

The methods and techniques described herein, in relation to determining the InfActPrimitive dataset, enhance the field of infant action recognition by highlighting the challenges specific to this domain, which has been largely unexplored despite the successes in adult action recognition. The limitations in available infant data necessitate the identification of new action categories that cannot be learned from existing datasets. To address this issue, the methods and techniques described herein focus on adapting action recognition models trained on adult data for use on infant action data, considering the adult-to-infant shift, and employing data-efficient methods.

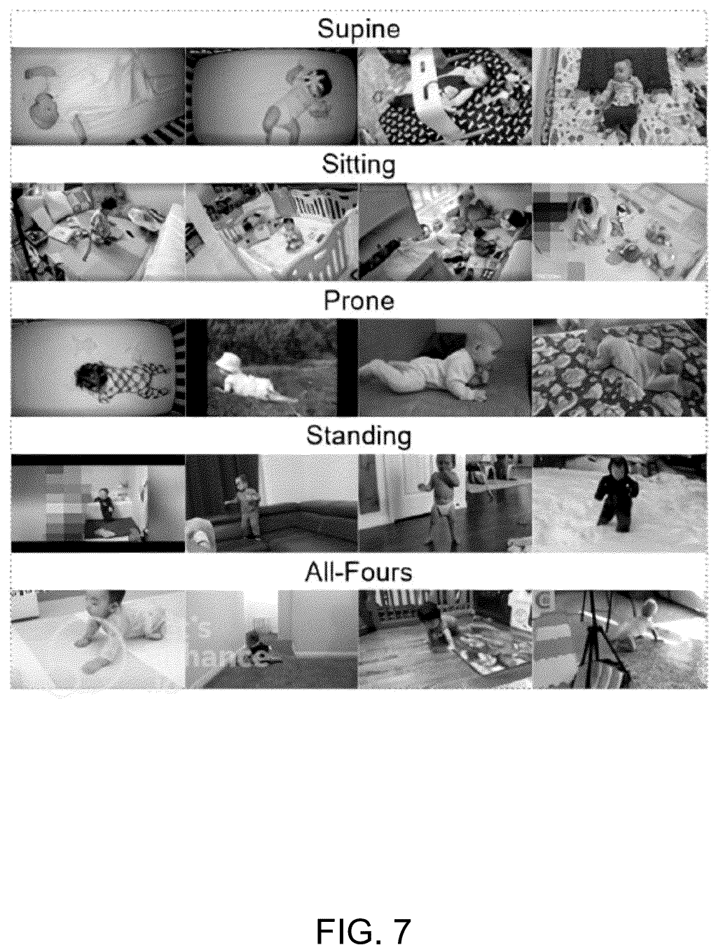

In summary, the methods and techniques described herein introduces a novel dataset called infant action (InfActPrimitive) specifically designed for studying infant action recognition. FIG. 7 illustrates some snapshots of InfActPrimitive. This dataset includes five motor primitive infant milestones as basic actions. Baseline experiments were conducted on the InfActPrimitive dataset using state-of-the-art skeleton-based action recognition models. These experiments provide a benchmark for evaluating the performance of infant action recognition algorithms. The methods and techniques described herein in determining the InfActPrimitive dataset provide insight into the challenges of adapting action recognition models from adult data to infant data. The determination of the InfActPrimitive dataset includes domain adaptation challenges and their practical implications for infant motor developmental monitoring, as well as general infant health and safety. Overall, these contributions enhance the understanding of infant action recognition and provide valuable resources for further research in this domain.

Vision-based human action recognition may be classified into different categories based on the type of input data, applications, model architecture, and techniques employed. The methods and techniques described herein focus on studies conducted specifically related to skeleton data (e.g., 2D or 3D body poses) in human action recognition. Additionally, the methods and techniques described herein apply vision-based approaches to the limited availability of infant data.

Recurrent neural network structures methods, such as long short term memory (LSTM) and gated recurrent unit (GRU), treat the skeleton sequences as sequential vectors, focusing primarily on capturing temporal information. However, these methods often overlook the spatial information present in the skeletons [63]. A part-aware LSTM model is introduced that utilizes separate stacked LSTMs for processing different groups of body joints, with the final output obtained through a dense layer combination, enhancing action recognition by capturing spatiotemporal patterns [71]. A global context-aware attention LSTM (GCA-LSTM) is proposed that incorporates a recurrent attention mechanism that selectively emphasizes the informative joints within each frame [65].

Graph convolutional network (GCN) has emerged as a prominent method for skeleton-based action recognition. GCN enables the efficient representation of spatiotemporal skeleton data by encapsulating the intricate nature of an action into a sequence of interconnected graphs. Spatial temporal graph convolution network (ST-GCN) introduced inter-inframe edges, connecting corresponding joints across consecutive frames. This approach enhances the modeling of inter-frame relationships and improves the understanding of temporal dynamics within the skeletal data. InfoGCN combines a learning objective and an encoding method using attention-based graph convolution that captures discriminative information of human actions.

3D convolutional networks capture the spatio-temporal information in skeleton sequences using image-based representations. Joint trajectories may be encoded into texture images using hue, saturation, and value (HSV) space, but the model performance may suffer from trajectory overlapping and the loss of past temporal information [76]. This issue is addressed by encoding pair-wise distances of skeleton joints into texture images and representing temporal information through color variations [62]. However, this model encountered difficulties in distinguishing actions with similar distances.

Available datasets for human action recognition mainly incorporate RGB videos with 2D/3D skeletal pose annotations. The majority of the aforementioned studies employed large labeled skeleton-based datasets, such as NTU RGB+D [71], which consists of over 56,000 sequences and 4 million frames, encompassing sixty different action classes. The Northwestern-UCLA (N-UCLA) is another widely used skeleton based dataset consisting of 1494 video clips featuring ten volunteers, captured using three Microsoft™ Kinect cameras from multiple angles to obtain 3D skeletons with twenty joints, encompassing a total of ten action categories.

Infant-specific computer vision studies have been relatively scarce while there have been notable advancements in computer vision within the adult domain. The majority of these studies have been primarily focused on infant images for tasks such as pose estimation [56, 80], facial landmarks detection [73, 81], posture classification [57, 59], and 3D synthetic data generation [67]. A VGG-16 model pretrained with adult faces is finetuned for infant facial action unit recognition [69]. The VGG-16 model is a convolutional neural network (CNN) architecture that was proposed by the Visual Geometry Group (VGG) at the University of Oxford. These methods are applied to the Craniofacial microsomia: Longitudinal Outcomes in Children pre-Kindergarten (CLOCK) dataset [55] and the “MIAMI” datasets [50], which were specifically designed to investigate neurodevelopmental and phenotypic outcomes in infants with craniofacial microsomia and assess the facial actions of 4-month-old infants in response to their parents, respectively. A CNN-based pipeline is proposed to detect and temporally segment the non-nutritive sucking pattern using nighttime in-crib baby monitor footage [81]. BabyNet, network structure aimed at infant reaching action recognition, that uses a residual network (ResNet) model followed by an LSTM to capture the spatial and temporal connection of annotated bounding boxes to interpret the onset and offset of reaching and to detect a complete reaching action [81]. However, the focus of these studies has predominantly been on a limited set of facial actions or the detection of specific actions, thereby neglecting actions that involve diverse poses and postures. This issue may be addressed by creating a small dataset containing a diverse range of infant actions and a few samples for each action [58]. A posture classification model was developed that was applied on every frame of an input video to extract the posture probability signal. Subsequently, a bi-directional LSTM is employed to segment the signal and estimate posture transitions and the action associated with that transition. Despite presenting a challenging dataset, this action recognition pipeline is not an end-to-end approach.

The methods and techniques described herein enhance the existing dataset (e.g., the small dataset containing a diverse range of infant actions) to create a more robust dataset. This expansion involves classifying actions into specific simple primitive motor actions, including “sitting,” “standing,” “prone,” “supine,” and “all-fours.” Additional video clips of infants in their natural environment were collected, encompassing both daytime play and nighttime rest, in various settings such as playtime and crib environments. Further, the intricate task of infant action recognition is contemplated through a comprehensive end-to-end approach, with a specific focus on the challenges associated with adapting action recognition models from the adult domain to the unique infant domain.

The goal of a human action recognition framework is to assign labels to the actions present in a given video. In the infant domain, the focus may be for the most common actions that are related to infant motor development milestones. The following is an introduction to the dataset and pipeline for modeling infant skeleton sequences, which aims to create distinct representations for infant action recognition. The dataset introduced, (e.g., the InfActPrimitive dataset), serves as the foundation for training and evaluating the pipeline. Subsequently, the details of the pipeline encompasses the entire process from receiving video frames as input to predicting infant action.

### The InfActPrimitive Dataset

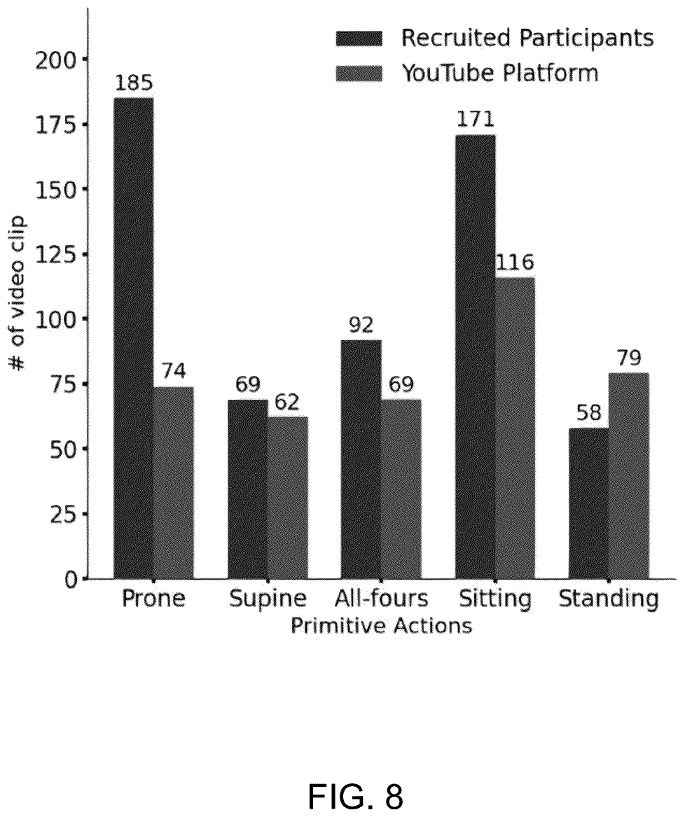

The methods and techniques described herein introduce a new dataset called InfActPrimitive as a benchmark to evaluate infant action recognition models. Videos in InfActPrimitive may be provided from multiple sources. Video sources may include videos submitted by recruited participants and/or videos gathered from public-sharing platforms (e.g., YouTube). Videos submitted by recruited participants may include videos collected of infant videos using a baby monitor from participants home and in an unscripted manner. For example, the experiment was approved by the Committee on the Use of Humans as Experimental Subjects of Northeastern University (Institutional Review Board (IRB) number: 22-11-32). Participants provided informed written consent before the experiment and were compensated for their time. Videos gathered from public video-sharing platforms may be acquired by performing searches for public videos on the YouTube platform. InfActPrimitive contains 814 infant action videos of five basic motor primitives representing specific postures such as sitting, standing, prone, supine, and all four. The start and end time of every motor primitive is meticulously annotated in this dataset. The InfActPrimitive, with its motor primitives defined by the Alberta Infant Motor Scale (AIMS) as significant milestones, is ideal for developing and testing models for infant action recognition, milestone tracking, and detection of complex actions. FIG. 7 illustrates sample screenshots from various videos within the InfActPrimitive dataset, illustrating the diversity of pose, posture, and action among the samples. The diverse range of infant ages and a wide variety of movements and postures within the InfActPrimitive dataset pose significant challenges for action recognition tasks. FIG. 8 shows the statistical analysis of InfActPrimitive for each sources of data separately.

### The Infant Action Recognition Pipeline

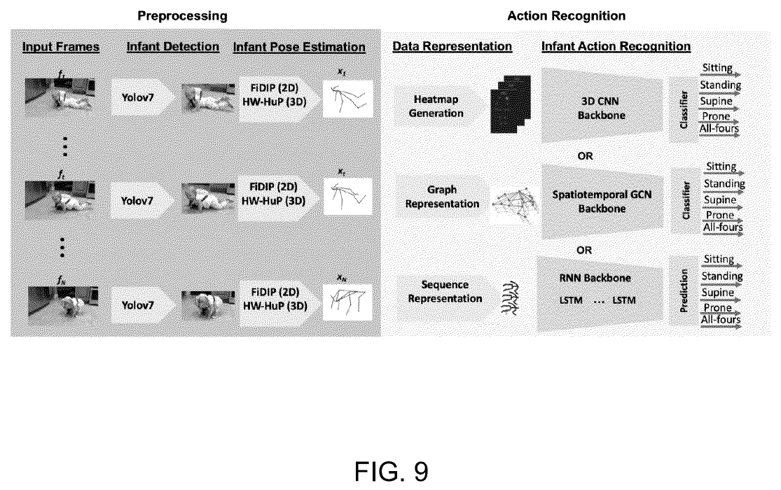

Infant specific prepossessing, skeleton data prediction, and action recognition are the key components of the Infant Action Recognition Pipeline (“the pipeline”), as shown in FIG. 9. To achieve this, input frames are processed through the pipeline's components, enabling infant-specific skeleton data generation and alignment as input to the different state-of-the-art action recognition models.

Preprocessing of the pipeline-Input video V is represented as a sequence of T frames, V=(f¹, . . . ,f^{t}, . . . , f^{T}). An object detection algorithm, such as You Only Look Once (YOLO) (e.g., YOLOv7 [74]), may be customized to locate the bounding box around the infants at every frame as a region of interest. A 2D or 3D infant skeleton pose prediction may be extracted, x^{t}∈ [symbol] ^{J×D}, where J=17 is the number of skeleton joints (corresponding to the shoulders, elbows, wrists, hips, knees, and ankles), and D∈{2, 3} is spatial dimension of the coordinates. The underlying pose estimators-the fine—tuned domain-adapted infant pose (FiDIP) model [56] for 2D and the heuristic weakly supervised 3D human pose estimation infant (HW-HuP-Infant) model [66] for 3D were specifically adapted for the infant domain.

Infant-adult skeleton alignment—One of the major challenges in the domain of skeleton-based action recognition lies in the significant variability of skeleton layouts across different datasets and scenarios. The diversity in joint definitions, proportions, scaling, and pose configurations across these layouts introduces complexity that directly impacts the efficacy of action recognition algorithms and makes transferring knowledge between two different datasets inefficient. The challenge of reconciling these layout differences and enabling robust recognition of actions regardless of skeletal variations is a critical concern in pose recognition.

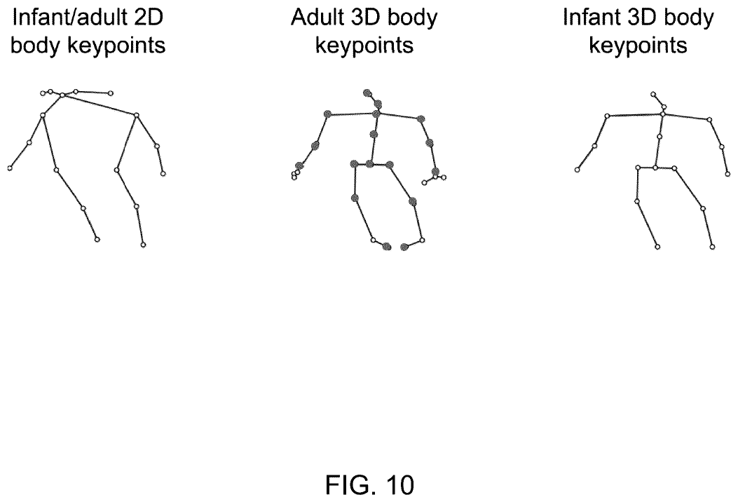

As shown in FIG. 10, the adult action dataset NTU RGB+D indicates the location of 25 joints in a 3D space. The layout of infant 3D skeletons in the InfActPrimitive dataset on the other hand, is based on the Human3.6M skeleton structure, which supports a total of 17 joints. To match the number of keypoints and align the skeleton data in these two datasets, a subset of joints of NTU RGB+D skeleton that are common with the Human3.6M layout are selected. The selected joints are also reordered so that the structures are as similar as possible. For the 2D skeletons, layouts of both NTU RGB+D dataset and InfActPrimitive dataset are based on the Common Objects in Context (COCO) structure.

Action recognition—After preprocessing, the extracted sequence of body keypoints from the input video is fed into various state-of-the-art skeleton-based action recognition models leveraging different aspects of infant-specific pose representations. These skeleton-based models may be categorized into three groups: CNN-based, graph-based, and RNN-based models so that the models may fully exploit the information encoded in the pose data and perform a comprehensive comparative analysis of the results.

Recurrent neural network structures capture the long-term temporal correlation of spatial features in the skeleton. A part-aware LSTM (PLSTM) [17] is applied to segment body joints into five part groups and use independent streams of LSTMs to handle each part. At each timeframe t, the input x^{t} is broken into (x₁^{t}, . . . , xₚ^{t}) parts, corresponding to P parts of the body. These inputs are fed into P streams of LSTM modules, where each LSTM has its own individual input, forget, and modulation gates. However, the output gate of these streams will be concatenated and will be shared among the body parts and their corresponding LSTM streams.

GCNs represent skeletal data as a graph structure, with joints as nodes and connections as edges. To capture temporal relationships, an ST-GCN is applied, which considers interframe connections between the same joints in consecutive frames. Furthermore, an InfoGCN is used, which integrates a spatial attention mechanism to understand context-dependent joint topology, enhancing the existing skeleton structure. The InfoGCN utilizes an encoder with graph convolutions and attention mechanisms to infer class-specific characteristics μ_{c} and diagonal covariance matrix of a multivariate Gaussian distribution σ_{c}, with an auxiliary independent random noise ϵ˜N(0, I), Z is sampled as Z=μ_{c}+Σ_{c}ϵ. The decoder block of the model, composed of a single linear layer and a softmax function, converts the latent vector Z to the categorical distribution.

3D convolutional networks are mainly employed in RGB-based action recognition tasks to capture both spatial and temporal features across consecutive frames. To utilize the capabilities of a CNN-based framework, keypoints in each frame are first converted into heatmaps. These heatmaps are generated by creating Gaussian maps centered at each joint within the frame. Subsequently, skeleton-based action recognition (e.g., the PoseC3D method) is applied, which involved stacking these heatmaps along the temporal dimension, enabling 3D-CNNs to effectively handle skeleton-based action detection. Lastly, the representations extracted from each input sequence using the 3D convolutional layer are fed into a classifier. This classifier consists of a single linear layer followed by a softmax function, ultimately yielding the final class distribution.

The Experimental results include an assessment of the performance of the models presented in the pipeline. This begins by providing an overview of the experimental setup and the datasets employed. Subsequently, there are multiple outcomes of the various experiments. Finally, ablation studies are conducted and delve into potential avenues for future enhancements.

### Evaluation Datasets

NTU RGB+D is a large-scale action recognition dataset with both RGB frames and 3D skeletons. This dataset contains 56,000 samples across 60 action classes. Video samples for this dataset have been captured by three Microsoft™ Kinect V2 camera sensors concurrently. 3D skeletal data contains the 3D locations of 25 major body joints at each frame. High-Resolution Net (HRNet), a general purpose convolutional neural network for tasks like semantic segmentation, object detection, and image classification, is used to estimate the 2D pose, which results in the coordination of 17 joints in the 2D space. Given that each video in this dataset features a minimum of two subjects, the approach involves evaluating the models within a cross-subject setting. In this particular setup, the models are trained using samples drawn from a designated subset of actors, while the subsequent evaluation is carried out on samples featuring actors who were not part of the training process. A train-test split paradigm is used that mirrors the methodology outlined in the determination of the NTU RGB+D Dataset. Specifically, the initial cohort of 40 subjects is partitioned into distinct training and testing groups, with each group composed of 20 subjects. In the context of this evaluative exercise, both the training and testing sets encompass a substantial number of samples, totaling 40, 320, and 16,560, respectively. It is noteworthy that the training subjects for this particular evaluation bear the following identification numbers: 1, 2, 4, 5, 8, 9, 13, 14, 15, 16, 17, 18, 19, 25, 27, 28, 31, 34, 35, and 38. The remaining subjects have been thoughtfully reserved for the purpose of conducting rigorous testing.

InfActPrimitive, as detailed above, combines video clips from two primary sources: data collected from the YouTube platform and data acquired through independent data collection efforts. To evaluate the performance of the pipeline on this dataset, the training set comprises all videos collected from YouTube, totaling 116 sitting, 79 standing, 62 supine, 74 prone, and 69 all-fours actions. Similarly, the test set consists exclusively of videos from the independently collected data, including 171 clips for sitting, 58 clips for standing, 62 clips for supine, 185 clips for prone, and 92 clips for all fours. This partitioning strategy enables assessment of the pipeline's ability to generalize across previously unobserved data and diverse sources, ensuring a comprehensive representation of various actions in both the training and test sets. This approach enhances the robustness of the evaluation by encompassing a wide range of settings and conditions found in the YouTube videos and the collected data.

### Experimental Setup

A series of experiments were conducted using the infant action recognition pipeline. A comparative analysis is also provided, examining the outcomes in relation to the adult skeleton data.

Baseline experiment—The baseline experiment includes trained various action recognition models, as detailed above, separately on both the NTU RGB+D and InfActPrimitive datasets from scratch. With the exception of PoseC3D, all these models established baseline performance levels for both 2D and 3D-based action recognition tasks across both adult and infant domains. This baseline performance provides a starting point against which the performance of future experiments, such as fine-tuning or incorporating domain-specific knowledge, can be compared. The hyperparameter for ST-GCN, InfoGCN, deepLSTM and PoseC3D models are set as originally specified [51, 53, 78]. In Table 5, the first pair of columns illustrate the experimental findings with 2D skeleton sequences from both the NTU RGB+D and InfActPrimitive datasets, respectively. Simultaneously, the fourth and fifth columns present the results in the context of 3D data. As demonstrated, PoseC3D consistently outperforms other models in both adult and infant action recognition domains. Nevertheless, a significant performance gap persists between infant and adult action recognition, which can be attributed to disparities in sample size and class distribution. The adult model benefits from a more abundant dataset, enabling it to effectively capture the spatiotemporal nuances of various actions, a characteristic that the InfActPrimitive dataset lacks.

| | Based on 2D Pose | | | Based on 3D Pose | | |
|---|---|---|---|---|---|---|
| Action Model | NTU RGB + D | InfActPrimitive | InfActPrimitive (+FT) | NTU RGB + D | InfActPrimitive | InfActPrimitive (+FT) |
| DeepLSTM [symbol] | 87.0 | 24.3 | 17.2 | x | x | x |
| ST-GCN [symbol] | 81.5 | 64.0 | 66.9 | 82.5 | 67.1 | 69.7 |
| InfoGCN [symbol] | 91.0 | 29.7 | 29.7 | 85.0 | 29.7 | 29.7 |
| PoseC3D [symbol] | 94.1 | 66.9 | 69.7 | x | x | x |

[symbol]

indicates data missing or illegible when filed

Table 5. Results of 2D/3D skeleton-based action recognition models using the pipeline on both adult (NTU RGB+D) and infant (InfActPrimitive) datasets. FT denotes that the model was pre-trained on NTU RGB+D during the transfer learning experiments. PoseC3D achieves the best performance on 2D data in both adult and infant datasets. PoseC3D only supports 2D data, and the results in 3D space are marked with X. The DeepLSTM model also resulted in unsatisfactory performance when applied to 3D skeleton data, which is denoted with X.

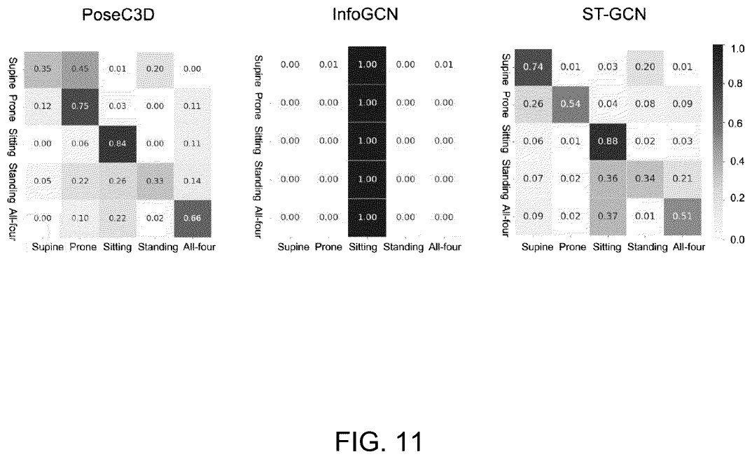

FIG. 11 shows the confusion matrices for PoseC3D, InfoGCN, and ST-GCN methods. As shown in FIG. 11, the sequences associated with the “sitting” action class exhibit superior separability compared to other classes. However, it is evident that the InfoGCN model fails in the infant action recognition.

Transfer learning experiment—To utilize the knowledge embedded in the adult action recognition, the model weights are initialized using the learned parameters obtained from prior training on the NTU RGB+D dataset. To address the substantial class disparities between the two datasets, the classifier weights are excluded, and for this experiment, initialized randomly.

Given the significant disparity in the number of classes between the two datasets and the substantial impact of training set size on model performance, there are greater implications of this experimental parameter. Notably, limited data availability posed challenges to achieving high accuracy in models trained on InfActPrimitive. To determine whether this issue extended beyond the domain of infant action recognition, modifications were made to the training subset of NTU RGB+D. Specifically, a subset was curated comprising only five action classes, namely, ‘sit down,’ ‘stand up,’ ‘falling down,’ ‘jump on,’ and ‘drop,’ which closely matched those in InfActPrimitive. The number of samples per class in this subset is restricted to align with the size of the InfActPrimitive training subset. The validation samples for these selected classes remained unchanged.

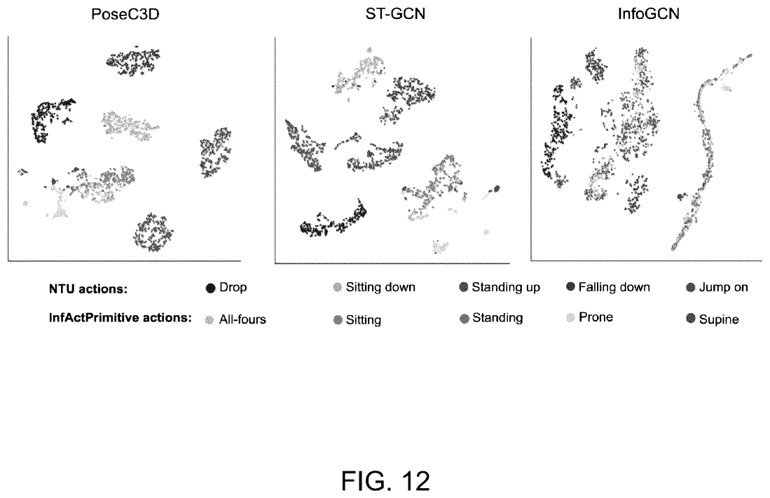

As shown in FIG. 12, the latent variables demonstrate a significantly greater degree of separability within the adult domain compared to the infant domain. This finding highlights the potential limitations of models pretrained on infants in capturing the underlying patterns specific to the infant domain. The disparity can be attributed to the substantial differences between the adult and infant domains, emphasizing the necessity for domain-specific model adaptations or training approaches.

Intra-class data diversity experiment—In the final experiment, the impact of intra-class diversity on action recognition model performance is investigated. The hypothesis is that the absence of structural coherence and the inherent variations among samples from the same class can significantly reduce validation accuracy. While traditional action recognition datasets like NTU RGB+D are known for rigid action instructions and minimal intra-class variation, the InfActPrimitive dataset, derived from in-the-wild videos, exhibits a higher level of variability in performed actions. To test this hypothesis, cross-validation training was conducted, dividing the training dataset into five subsets and training on four while validating on the fifth. The original validation set of InfActPrimitive was used for testing. Given the superior results achieved with the PoseC3D model using 2D skeleton data, this model was considered as an infant action recognition model. The findings are presented in Table 6, which shed light on the influence of intra-class diversity on action recognition model performance.

TABLE 6
Infant action recognition results with inter-class data diversity
using PoseC3D [53]. InfActPrimitive training set is partitioned
into five folds, with one fold reserved for validation while the
remaining folds were used to train the model. The last row of the
table presents the mean and variance computed across all folds.

| Held-out fold | Train | Validation | Test |
|---|---|---|---|
| Fold 1 | 93.7 | 83.7 | 64.3 |
| Fold 2 | 87.5 | 91.2 | 61.2 |
| Fold 3 | 93.7 | 83.0 | 56.3 |
| Fold 4 | 93.7 | 78.7 | 60.8 |
| Fold 5 | 93.7 | 85.0 | 50.6 |
| Average | 92.50 ± 6.2 | 84.3 ± 16.3 | 58.6 ± 22.7 |

As shown in Table 6, although each experiment yields high training accuracy, there are substantial variations in validation and testing accuracies across experiments. These outcomes reveal discrepancies in the training datasets, leading to inconsistent learning, and underscore distinctions between videos collected from diverse sources.

The InfActPrimitive dataset introduces a unique dataset for infant action recognition, which may serve as an invaluable benchmark for the field of infant action recognition and milestone tracking. Through this research, state-of-the-art skeleton-based action recognition techniques were applied, with Pose3D achieving reasonable performance. However, it is important to note that most other successful state-of-the-art action recognition methods failed when it came to categorizing infant actions. This stark contrast underscores a significant knowledge gap between infant and adult action recognition modeling. This divergence arises from the distinct dynamics inherent in infant movements compared to those of adults, emphasizing the need for specialized, data-efficient models tailored explicitly for infant video datasets. Addressing this challenge is crucial to advancing the field of infant action recognition and ensuring that the developmental milestones of the youngest subjects are accurately tracked and understood. These findings shed light on the unique intricacies of infant actions and pave the way for future research to bridge the gap in modeling techniques and foster a deeper understanding of infant development.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to 10 effect various changes, substitutions of equivalents, and other alterations to the compositions and methods set forth herein.

### REFERENCES

[1] Yossi Adi, Joseph Keshet, Emily Cibelli, and Matthew Goldrick. Sequence segmentation using joint rnn and structured prediction models. In 2017 IEEE International Conference on Acoustics, Speech and Signal Processing (ICASSP), pages 2422-2426. IEEE, 2017.

[2] Abid Ali, Farhood F Negin, Francois F Bremond, and Susanne Thümmler. Video-based behavior understanding of children for objective diagnosis of autism. In VISAPP 2022-17th International Conference on Computer Vision Theory and Applications, 2022.

[3] Mykhaylo Andriluka, Leonid Pishchulin, Peter Gehler, and Bernt Schiele. 2d human pose estimation: New benchmark and state of the art analysis. In Proceedings of the IEEE Conference on computer Vision and Pattern Recognition, pages 3686-3693, 2014.

[4] Xu Cao, Xiaoye Li, Liya Ma, Yi Huang, Xuan Feng, Zen-ing Chen, Hongwu Zeng, and Jianguo Cao. Aggpose: Deep aggregation vision transformer for infant pose estimation. In Proceedings of the Thirty-First International Joint Conference on Artificial Intelligence (IJCAI-22) Special Track on Alfor Good, 2022.

[5] Rizwan Chaudhry, Avinash Ravichandran, Gregory Hager, and Rene\acute{ } Vidal. Histograms of oriented optical flow and binet-cauchy kernels on nonlinear dynamical systems for the recognition of human actions. In 2009 IEEE conference on computer vision and pattern recognition, pages 1932-1939. IEEE, 2009.

[6] Chen Chen, Kui Liu, and Nasser Kehtarnavaz. Real-time human action recognition based on depth motion maps. Journal of real-time image processing, 12:155-163, 2016.

[7] Marco Cristani, Ramachandra Raghavendra, Alessio Del Bue, and Vittorio Murino. Human behavior analysis in video surveillance: A social signal processing perspective. Neurocomputing, 100:86-97, 2013.

[8] Ryan Anthony J de Belen, Tomasz Bednarz, Arcot Sowmya, and Dennis Del Favero. Computer vision in autism spectrum disorder research: a systematic review of published stud-ies from 2009 to 2019. Translational psychiatry, 10(1):333, 2020.

[9] Amel Dechemi, Vikarn Bhakri, Ipsita Sahin, Arjun Modi, Julya Mestas, Pamodya Peiris, Dannya Enriquez Barrun-dia, Elena Kokkoni, and Konstantinos Karydis. Babynet: A lightweight network for infant reaching action recognition in unconstrained environments to support future pediatric rehabilitation applications. In 2021 30th IEEE International Conference on Robot & Human Interactive Communication (RO-MAN), pages 461-467. IEEE, 2021.

[10]

Kimberly A Fournier, Chris J Hass, Sagar K Naik, Neha Lodha, and James H Cauraugh. Motor coordination in autism spectrum disorders: a synthesis and meta-analysis. Journal of autism and developmental disorders, 40:1227-1240, 2010.

[11] David Gerónimo and Hedvig Kjellström. Unsupervised surveillance video retrieval based on human action and appearance. In 2014 22nd International Conference on Pattern Recognition, pages 4630-4635. IEEE, 2014.

[12] Nikolas Hesse, Christoph Bodensteiner, Michael Arens, Ulrich G Hofmann, Raphael Weinberger, and A Sebastian Schroeder. Computer vision for medical infant motion analysis: State of the art and rgb-d data set. In Proceedings of the European Conference on Computer Vision (ECCV) Workshops, pages 0-0, 2018.

[13] Cheng-Ming Huang, Yi-Ru Chen, and Li-Chen Fu. Real-time object detection and tracking on a moving camera platform. In 2009 ICCAS-SICE, pages 717-722. IEEE, 2009.

[14] Xiaofei Huang, Nihang Fu, Shuangjun Liu, and Sarah Ostadabbas. Invariant representation learning for infant pose estimation with small data. In 2021 16th IEEE International Conference on Automatic Face and Gesture Recognition (FG 2021), pages 1-8. IEEE, 2021.

[15] Xiaofei Huang, Shuangjun Liu, Michael Wan, Nihang Fu, David Pino, Bharath Modayur, and Sarah Ostadabbas. Appearance-independent pose-based posture classification in infants. In ICPR T-CAP Workshops, 2022.

[16] Xiaofei Huang, Michael Wan, Lingfei Luan, Bethany Tunik, and Sarah Ostadabbas. Computer vision to the rescue: Infant postural symmetry estimation from incongruent annotations. In Proceedings of the IEEE/CVF Winter Conference on Applications of Computer Vision, pages 1909-1917, 2023.

[17] Jana M Iverson and Mary K Fagan. Infant vocal-motor coordination: precursor to the gesture-speech system? Child development, 75(4):1053-1066, 2004.

[18] Jana M Iverson and Esther Thelen. Hand, mouth and brain. the dynamic emergence of speech and gesture. Journal of Consciousness studies, 6(11-12):19-40, 1999.

[19] Jungseock Joo, Erik P Bucy, and Claudia Seidel. Automated coding of televised leader displays: Detecting nonverbal political behavior with computer vision and deep learning. International Journal of Communication (19328036), 2019.

[20] Muhammad Attique Khan, Kashif Javed, Sajid Ali Khan, Tanzila Saba, Usman Habib, Junaid Ali Khan, and Aaqif Afzaal Abbasi. Human action recognition using fusion of multiview and deep features: an application to video surveillance. Multimedia tools and applications, pages 1-27, 2020.

[21] Ivan Laptev, Marcin Marszalek, Cordelia Schmid, and Benjamin Rozenfeld. Learning realistic human actions from movies. In 2008 IEEE Conference on Computer Vision and Pattern Recognition, pages 1-8. IEEE, 2008.

[22] Hayley C Leonard and Elisabeth L Hill. The impact of motor development on typical and atypical social cognition and language: A systematic review. Child and Adolescent Mental Health, 19(3):163-170, 2014.

[23] Chang Li, Qian Huang, Xing Li, and Qianhan Wu. Human action recognition based on multi-scale feature maps from depth video sequences. Multimedia Tools and Applications, 80:32111-32130, 2021.

[24] Tsung-Yi Lin, Michael Maire, Serge Belongie, James Hays, Pietro Perona, Deva Ramanan, Piotr Dolla\acute{ }r, and C Lawrence Zitnick. Microsoft coco: Common objects in context. In Computer Vision-ECCV 2014: 13th European Conference, Zurich, Switzerland, Sep. 6-12, 2014, Proceedings, Part V 13, pages 740-755. Springer, 2014.

[25] Shuangjun Liu, Xiaofei Huang, Nihang Fu, and Sarah Ostadabbas. Heuristic weakly supervised 3d human pose estimation in novel contexts without any 3d pose ground truth. arXiv preprint arXiv:2105.10996, 2021.

[26] Lucia Migliorelli, Sara Moccia, Rocco Pietrini, Virgilio Paolo Carnielli, and Emanuele Frontoni. The babypose dataset. Data in brief, 33:106329, 2020.

[27] Fabian Nater, Helmut Grabner, and Luc Van Gool. Exploiting simple hierarchies for unsupervised human behavior analysis. In 2010 IEEE Computer Society Conference on Computer Vision and Pattern Recognition, pages 2014-2021. IEEE, 2010.

[28] Juan Carlos Niebles and Li Fei-Fei. A hierarchical model of shape and appearance for human action classification. In 2007 IEEE Conference on computer vision and pattern recognition, pages 1-8. IEEE, 2007.

[29] Martha Piper and Johanna Darrah. Motor Assessment of the Developing Infant-E-Book: Alberta Infant Motor Scale (AIMS). Elsevier Health Sciences, 2021. 2, 4

[30] Hossein Rahmani and Mohammed Bennamoun. Learning action recognition model from depth and skeleton videos. In Proceedings of the IEEE international conference on computer vision, pages 5832-5841, 2017.

[31] Sam Roweis. Em algorithms for pca and spca. Advances in neural information processing systems, 10, 1997.

[32] Leah Sack, Christine Dollaghan, and Lisa Goffman. Contributions of early motor deficits in predicting language outcomes among preschoolers with developmental language disorder. International journal of speech-language pathology, 24(4):362-374, 2022.

[33] Adrian Sanchez-Caballero, David Fuentes-Jimenez, and Cristina Losada-Gutie\acute{ }rrez. Exploiting the convlstm: Human action recognition using raw depth video-based recurrent neural networks. arXiv preprint arXiv:2006.07744, 2020.

[34] Amir Shahroudy, Jun Liu, Tian-Tsong Ng, and Gang Wang. Ntu rgb+d: A large scale dataset for 3d human activity analysis. In Proceedings of the IEEE conference on computer vision and pattern recognition, pages 1010-1019, 2016.

[35] Ahmed Snoun, Nozha Jlidi, Tahani Bouchrika, Olfa Jemai, and Mourad Zaied. Towards a deep human activity recognition approach based on video to image transformation with skeleton data. Multimedia Tools and Applications, 80:29675-29698, 2021.

[36] Zehua Sun, Qiuhong Ke, Hossein Rahmani, Mohammed Bennamoun, Gang Wang, and Jun Liu. Human action recog¬nition from various data modalities: A review. IEEE trans-actions on pattern analysis and machine intelligence, 2022.

[37] Pieter Vanneste, Jose\acute{ }Oramas, Thomas Verelst, Tinne Tuytelaars, Annelies Raes, Fien Depaepe, and Wim Van den Noortgate. Computer vision and human behaviour, emotion and cognition detection: A use case on student engagement. Mathematics, 9(3):287, 2021.

[38] Kathan Vyas, Rui Ma, Behnaz Rezaci, Shuangjun Liu, Michael Neubauer, Thomas Ploetz, Ronald Oberleitner, and Sarah Ostadabbas. Recognition Of Atypical Behavior in Autism Diagnosis from Video using Pose Estimation over Time. In IEEE 29th International Workshop on Machine Learning for Signal Processing (MLSP), pages 1-6, 2019.

[39] Michael Wan, Xiaofei Huang, Bethany Tunik, and Sarah Ostadabbas. Automatic assessment of infant face and upper-body symmetry as early signs of torticollis. arXiv preprint arXiv:2210.15022, 2022.

[40] Michael Wan, Shaotong Zhu, Lingfei Luan, Prateek Gu-lati, Xiaofei Huang, Rebecca Schwartz-Mette, Marie Hayes, Emily Zimmerman, and Sarah Ostadabbas. InfAnFace: Bridging the infant-adult domain gap in facial landmark estimation in the wild. In 2022 International Conference on Pattern Recognition (ICPR), 2022.

[41] Haoran Wang, Baosheng Yu, Kun Xia, Jiaqi Li, and Xin Zuo. Skeleton edge motion networks for human action recognition. Neurocomputing, 423:1-12, 2021.

[42] Jiang Wang, Xiaohan Nie, Yin Xia, Ying Wu, and Song-Chun Zhu. Cross-view action modeling, learning and recognition. In Proceedings of the IEEE conference on computer vision and pattern recognition, pages 2649-2656, 2014.

[43] Di Wu, Nabin Sharma, and Michael Blumenstein. Recent advances in video-based human action recognition using deep learning: A review. In 2017 International Joint Conference on Neural Networks (IJCNN), pages 2865-2872. IEEE, 2017.

[44] Qingqiang Wu, Guanghua Xu, Fan Wei, Jiachen Kuang, Penglin Qin, Zejiang Li, and Sicong Zhang. Supine infant pose estimation via single depth image. IEEE Transactions on Instrumentation and Measurement, 71:1-11, 2022.

[45] Jie Xu, Rui Song, Haoliang Wei, Jinhong Guo, Yifei Zhou, and Xiwei Huang. A fast human action recognition network based on spatio-temporal features. Neurocomputing, 441:350-358, 2021.

[46] G Udny Yule. The applications of the method of correlation to social and economic statistics. Journal of the Royal Statistical Society, 72(4):721-730, 1909.

[47] M Zhdanova, V Voronin, E Semenishchev, Yu Ilyukhin, and A Zelensky. Human activity recognition for efficient human-robot collaboration. In Artificial Intelligence and Machine Learning in Defense Applications II, volume 11543, pages 94-104. SPIE, 2020.

[48] Jianxiong Zhou, Zhongyu Jiang, Jang-Hee Yoo, and Jenq-Neng Hwang. Hierarchical pose classification for infant action analysis and mental development assessment. In ICASSP 2021-2021 IEEE International Conference on Acoustics, Speech and Signal Processing (ICASSP), pages 1340-1344. IEEE, 2021.

[49] Shaotong Zhu, Michael Wan, Elaheh Hatamimajoumerd, Kashish Jain, Samuel Zlota, Cholpady Vikram Kamath, Cassandra B. Rowan, Emma C. Grace, Matthew S. Good-win, Marie J. Hayes, Rebecca A. Schwartz-Mette, Emily Zimmerman, and Sarah Ostadabbas. A Video-based End-to-end Process for Non-nutritive Sucking Action Recognition and Segmentation in Young Infants, March 2023. arXiv:2303.16867 [cs].

[50] Meng Chen, Sy-Miin Chow, Zakia Hammal, Daniel S Messinger, and Jeffrey F Cohn. A person-and time-varying vector autoregressive model to capture interactive infant-mother head movement dynamics. Multivariate behavioral research, 56(5):739-767, 2021.

[51] Hyung-gun Chi, Myoung Hoon Ha, Seunggeun Chi, Sang Wan Lec, Qixing Huang, and Karthik Ramani. Infogcn: Representation learning for human skeleton-based action recognition. In Proceedings of the IEEE/CVF Conference on Computer Vision and Pattern Recognition, pages 20186-20196, 2022.

[52] Amel Dechemi, Vikarn Bhakri, Ipsita Sahin, Arjun Modi, Julya Mestas, Pamodya Peiris, Dannya Enriquez Barrundia, Elena Kokkoni, and Konstantinos Karydis. Babynet: A lightweight network for infant reaching action recognition in unconstrained environments to support future pediatric rehabilitation applications. In 2021 30th IEEE International Conference on Robot & Human Interactive Communication (RO-MAN), pages 461-467, 2021.

[53] Haodong Duan, Yue Zhao, Kai Chen, Dahua Lin, and Bo Dai. Revisiting skeleton-based action recognition. In Proceedings of the IEEE/CVF Conference on Computer Vision and Pattern Recognition, pages 2969-2978, 2022.

[54] Rubia do N Fuentefria, Rita C Silveira, and Renato S Procianoy. Motor development of preterm infants assessed by the alberta infant motor scale: systematic review article. Jornal de pediatria, 93:328-342, 2017.

[55] Zakia Hammal, Wen-Sheng Chu, Jeffrey F Cohn, Carrie Heike, and Matthew L Speltz. Automatic action unit detection in infants using convolutional neural network. In 2017 Seventh International Conference on Affective Computing and Intelligent Interaction (ACII), pages 216-221. IEEE, 2017.

[56] Xiaofei Huang, Nihang Fu, Shuangjun Liu, and Sarah Ostadabbas. Invariant representation learning for infant pose estimation with small data. In 2021 16th IEEE International Conference on Automatic Face and Gesture Recognition (FG 2021), pages 1-8. IEEE, 2021.

[57] Xiaofei Huang, Shuangjun Liu, Michael Wan, Nihang Fu, Bharath Modayur, David Li Pino, and Sarah Ostadabbas. Appearance-independent pose-based posture classification in infants. In Workshop at the International Conference on Pattern Recognition (ICPRW), 8 2022.

[58] Xiaofei Huang, Lingfei Luan, Elahch Hatamimajoumerd, Michael Wan, Pooria Daneshvar Kakhaki, Rita Obeid, and Sarah Ostadabbas. Posture-based infant action recognition in the wild with very limited data. In Proceedings of the IEEE/CVF Conference on Computer Vision and Pattern Recognition, pages 4911-4920, 2023.

[59] Xiaofei Huang, Michael Wan, Lingfei Luan, Bethany Tunik, and Sarah Ostadabbas. Computer vision to the rescue: Infant postural symmetry estimation from incongruent annotations. In IEEE/CVF Winter Conference on Applications of Computer Vision (WACV), 1 2023.

[60] Alejandro Jaimes and Nicu Sebe. Multimodal human-computer interaction: A survey. Computer vision and image understanding, 108(1-2):116-134, 2007.

[61] Thomas N Kipf and Max Welling. Semi-supervised classification with graph convolutional networks. arXiv preprint arXiv: 1609.02907, 2016.

[62] Chuankun Li, Yonghong Hou, Pichao Wang, and Wanqing Li. Joint distance maps based action recognition with convolutional neural networks. IEEE Signal Processing Letters, 24(5):624-628, 2017.

[63] Chuankun Li, Pichao Wang, Shuang Wang, Yonghong Hou, and Wanqing Li. Skeleton-based action recognition using Istm and cnn. In 2017 IEEE International Conference on Multimedia & Expo Workshops (ICMEW), pages 585-590. IEEE, 2017.

[64] Guiyu Liu, Jiuchao Qian, Fei Wen, Xiaoguang Zhu, Rendong Ying, and Peilin Liu. Action recognition based on 3d skeleton and rgb frame fusion. In 2019 IEEE/RSJ International Conference on Intelligent Robots and Systems (IROS), pages 258-264. IEEE, 2019.

[65] Jun Liu, Gang Wang, Ling-Yu Duan, Kamila Abdiyeva, and Alex C Kot. Skeleton-based human action recognition with global context-aware attention Istm networks. IEEE Transactions on Image Processing, 27(4):1586-1599, 2017.

[66] Shuangjun Liu, Xiaofei Huang, Nihang Fu, and Sarah Ostadabbas. Heuristic weakly supervised 3d human pose estimation in novel contexts without any 3d pose ground truth. arXiv preprint arXiv: 2105.10996, 2021.

[67] Shuangjun Liu, Michael Wan, Xiaofei Huang, and Sarah Ostadabbas. Heuristic weakly supervised 3d human pose estimation in novel contexts without any 3d pose ground truth. arXiv, 2023.3

[68] Thomas B Moeslund, Adrian Hilton, and Volker Krüger. A survey of advances in vision-based human motion capture and analysis. Computer vision and image understanding, 104(2-3):90-126, 2006.

[69] Itir Onal Ertugrul, Yeojin Amy Ahn, Maneesh Bilalpur, Daniel S Messinger, Matthew L Speltz, and Jeffrey F Cohn. Infant afar: Automated facial action recognition in infants. Behavior research methods, 55(3):1024-1035, 2023.

[70] Behnaz Rezaei, Yiorgos Christakis, Bryan Ho, Kevin Thomas, Kelley Erb, Sarah Ostadabbas, and Shyamal Patel. Target-specific action classification for automated assessment of human motor behavior from video. Sensors, 19(19):4266, 2019.

[71] Amir Shahroudy, Jun Liu, Tian-Tsong Ng, and Gang Wang. Ntu rgb+d: A large scale dataset for 3d human activity analysis. In Proceedings of the IEEE conference on computer vision and pattern recognition, pages 1010-1019, 2016.

[72] Rajesh Kumar Tripathi, Anand Singh Jalal, and Subhash Chand Agrawal. Suspicious human activity recognition: a review. Artificial Intelligence Review, 50:283-339, 2018.

[73] Michael Wan, Shaotong Zhu, Lingfei Luan, Gulati Prateek, Xiaofei Huang, Rebecca Schwartz-Mette, Marie Hayes, Emily Zimmerman, and Sarah Ostadabbas. Infanface: Bridging the infant-adult domain gap in facial landmark estimation in the wild. In 2022 26th International Conference on Pattern Recognition (ICPR), pages 4486-4492. IEEE, 2022.

[74] Chien-Yao Wang, Alexey Bochkovskiy, and Hong-Yuan Mark Liao. Yolov7: Trainable bag-of-freebies sets new state-of-the-art for real-time object detectors. In Proceedings of the IEEE/CVF Conference on Computer Vision and Pattern Recognition (CVPR), pages 7464-7475 June 2023.

[75] Jiang Wang, Xiaohan Nie, Yin Xia, Ying Wu, and Song-Chun Zhu. Cross-view action modeling, learning and recognition. In Proceedings of the IEEE conference on computer vision and pattern recognition, pages 2649-2656, 2014.

[76] Pichao Wang, Zhaoyang Li, Yonghong Hou, and Wanqing Li. Action recognition based on joint trajectory maps using convolutional neural networks. In Proceedings of the 24^{th} ACM international conference on Multimedia, pages 102-106, 2016.

[77] Fei Wu, Qingzhong Wang, Jiang Bian, Ning Ding, Feixiang Lu, Jun Cheng, Dejing Dou, and Haoyi Xiong. A survey on video action recognition in sports: Datasets, methods and applications. IEEE Transactions on Multimedia, 2022.

[78] Sijie Yan, Yuanjun Xiong, and Dahua Lin. Spatial temporal graph convolutional networks for skeleton-based action recognition. In Proceedings of the AAAI conference on artificial intelligence, volume 32, 2018.

[79] Si Zhang, Hanghang Tong, Jiejun Xu, and Ross Maciejewski. Graph convolutional networks: a comprehensive review. Computational Social Networks, 6(1):1-23, 2019.

[80] Jianxiong Zhou, Zhongyu Jiang, Jang-Hee Yoo, and Jenq-Neng Hwang. Hierarchical pose classification for infant action analysis and mental development assessment. In ICASSP 2021-2021 IEEE International Conference on Acoustics, Speech and Signal Processing (ICASSP), pages 1340-1344, 2021.

[81] Shaotong Zhu, Michael Wan, Elaheh Hatamimajoumerd, Cholpady Vikram Kamath, Kashish Jain, Samuel Zlota, Emma Grace, Cassandra Rowan, Matthew Goodwin, Rebecca Schwartz-Mette, Emily Zimmerman, Marie Hayes, and Sarah Ostadabbas. A video-based end-to-end pipeline for non-nutritive sucking action recognition and segmentation in young infants. In International Conference on Medical Image Computing and Computer-Assisted Intervention (MICCAI), 10 2023.

What is claimed is:

1. A computer-implemented method for recognizing an infant action in a video recording, comprising:
receiving a video segment of said video recording that captures movement of an infant, wherein the video segment includes a plurality of frames;
determining, using a posture classification model, posture classification data representing a posture prediction for each frame of the plurality of frames;
determining a first subset of the plurality of frames representing a transition segment between two stable posture segments, wherein the transition segment includes a first frame in time and a last frame in time;
determining, based on the posture classification data and the first frame in time of the transition segment, a second subset of the plurality of frames representing a start posture segment;
determining, based on the posture classification data and the last frame in time of the transition segment, a third subset of the plurality of frames representing an end posture segment;
determining a start posture label for the start posture segment;
determining an end posture label for the end posture segment; and
determining, based on the start posture label and the end posture label, an infant action label for the video segment.

2. The computer-implemented method of claim 1, further comprising:
determining probability values corresponding to each frame of the plurality of frames and representing a confidence score for the posture prediction of the corresponding frame; wherein determining the first subset of the plurality of frames representing the transition segment further comprises:
determining a fourth subset of the plurality of frames representing a period of uncertainty, wherein the probability values of frames corresponding to the fourth subset fail to exceed a threshold value; and
determining the fourth subset corresponds with the first subset.

3. The computer-implemented method of claim 2, wherein determining the start posture label further comprises determining a first stable posture by performing majority voting of the probability values corresponding to the second subset;
wherein determining the end posture label further comprises determining a second stable posture by performing majority voting of the probability values corresponding to the third subset.

4. The computer-implemented method of claim 1, further comprising:
prior to determining the posture classification, determining, using a pose estimation model, pose estimation data representing a human skeleton pose for each frame of the plurality of frames, wherein the human skeleton pose is based on joint locations and joint angles of the infant; and
providing the pose estimation data as input to the posture classification model.

5. The computer-implemented method of claim 4, wherein the pose estimation model is trained using an adult pose dataset and an augmented dataset including real-world infant pose data and synthetic infant pose data.

6. The computer-implemented method of claim 4, wherein the posture classification model is trained using a two-dimensional infant pose dataset and a three-dimensional infant pose dataset.

7. The computer-implemented method of claim 4, wherein determining the first subset of the plurality of frames representing the transition segment further comprises:
extracting, based on the pose estimation data, a set of feature vectors corresponding to the plurality of frames;
determining, using a transition segmentor model with the set of feature vectors as input, the first subset, wherein the transition segmentor model is trained using vectors representing posture transitions.

8. The computer-implemented method of claim 7, wherein the set of feature vectors are extracted from a penultimate layer of the posture classification model.

9. The computer-implemented method of claim 1, wherein the posture classification model classifies a posture as one of supine, prone, sitting, standing, or all-fours.

10. A system for recognizing an infant action in a video recording, comprising:
at least one processor; and
at least one memory including instructions that, when executed by the at least one processor, cause the system to:
receive a video segment of said video recording that captures movement of an infant, wherein the video segment includes a plurality of frames;
determine, using a posture classification model, posture classification data representing a posture prediction for each frame of the plurality of frames;
determine a first subset of the plurality of frames representing a transition segment between two stable posture segments, wherein the transition segment includes a first frame in time and a last frame in time;
determine, based on the posture classification data and the first frame in time of the transition segment, a second subset of the plurality of frames representing a start posture segment;
determine, based on the posture classification data and the last frame in time of the transition segment, a third subset of the plurality of frames representing an end posture segment;
determine a start posture label for the start posture segment;
determine an end posture label for the end posture segment; and
determine, based on the start posture label and the end posture label, an infant action label for the video segment.

11. The system of claim 10, wherein the at least one memory further comprises instructions that, when executed by the at least one processor, further cause the system to:
determine probability values corresponding to each frame of the plurality of frames and representing a confidence score for the posture prediction of the corresponding frame; wherein determining the first subset of the plurality of frames representing the transition segment further includes instructions to:
determine a fourth subset of the plurality of frames representing a period of uncertainty, wherein the probability values of frames corresponding to the fourth subset fail to exceed a threshold value; and
determine the fourth subset corresponds with the first subset.

12. The system of claim 11, wherein determining the start posture label further comprises determining a first stable posture by performing majority voting of the probability values corresponding to the second subset;
wherein determining the end posture label further includes instructions to determine a second stable posture by performing majority voting of the probability values corresponding to the third subset.

13. The system of claim 10, wherein the at least one memory further comprises instructions that, when executed by the at least one processor, further cause the system to:
prior to determining the posture classification, determine, using a pose estimation model, pose estimation data representing a human skeleton pose for each frame of the plurality of frames, wherein the human skeleton pose is based on joint locations and joint angles of the infant; and
provide the pose estimation data as input to the posture classification model.

14. The system of claim 13, wherein the pose estimation model is trained using an adult pose dataset and an augmented dataset including real-world infant pose data and synthetic infant pose data.

15. The system of claim 13, wherein the posture classification model is trained using a two-dimensional infant pose dataset and a three-dimensional infant pose dataset.

16. The system of claim 13, wherein determining the first subset of the plurality of frames representing the transition segment further comprises instructions that, when executed by the at least one processor, further cause the system to:
extract, based on the pose estimation data, a set of feature vectors corresponding to the plurality of frames;
determine, using a transition segmentor model with the set of feature vectors as input, the first subset, wherein the transition segmentor model is trained using vectors representing posture transitions.

17. The system of claim 16, wherein the set of feature vectors are extracted from a penultimate layer of the posture classification model.

18. The system of claim 10, wherein the posture classification model classifies a posture as one of supine, prone, sitting, standing, or all-fours.

19. A computer-implemented method of generating a dataset of a plurality of infant actions, comprising:
receiving a plurality of video recordings that capture actions of human infants;
determining an infant action label for each video recording of the plurality of video recordings, wherein determining the infant action label for a video recording further comprises:
determining a region of interest for each frame of the video recording, wherein the region of interest corresponds to detection of an infant;
determining, using the region of interest for each frame, a skeletal pose;
determining, using the skeletal pose, a set of skeleton keypoints corresponding to an adult skeleton; and
determining, using an action recognition model with the set of skeleton keypoints as input, the infant action label;
labeling each video of the plurality of video recordings with the infant action label corresponding to the video recording; and
storing the plurality of videos labeled with the infant action label in a database.

20. The computer-implemented method of claim 19, wherein the action recognition model is one of:
(a) a recurrent neural network with the skeleton keypoints separated into body part groups;
(b) a graph convolutional network with the skeleton keypoints represented as a graph, wherein joints are nodes of the graph and connections between the joints are edges of the graph; and
(c) a three-dimensional convolutional network with the skeleton keypoints from each frame converted into a heatmap.
